(19) United States
(12) Patent Application Publication
(10) Pub. No.: US 2011/0306819 A1
(43) Pub. Date: Dec. 15, 2011

(54) Methods, Devices And Systems For Cardioelectromagnetic Treatment

(76) Inventors: Jerry I. Jacobson, Jupiter, FL (US); Benjamin J. Scherlag, Oklahoma City, OK (US); William S. Yamanashi, Edmond, OK (US); Allison Yamanashi Leib, Berkeley, CA (US)

(21) Appl. No.: 13/031,369

(22) Filed: Feb. 21, 2011

Related U.S. Application Data
(63) Continuation-in-part of application No. 11/711,524, filed on Feb. 27, 2007.
Application No. 11/711,524 is a division of application No. 10/682,131, filed on Oct. 9, 2003, now Pat. No. 7,186,209.

(51) Int. Cl. A61N 2/02

(52) U.S. Cl. 600/9

(57) ABSTRACT

Disclosed are methods of treatment or prophylaxis of a cardiac-related condition by electromagnetic field application. A person having or susceptible to such condition is subjected to low-level electromagnetic fields having a frequency between zero up to about 200 Hertz. The diseased state or condition may include diseased heart valves, an enlarged heart, circulatory blockage, coronary insufficiencies, and ischemia. The treatment may be administered non-invasively or invasively. An implantable device for invasively administering the treatment may include at least one component emitting electromagnetic fields having a frequency between zero and about 200 Hertz. The component may include at least one inductor.

### PRIORITY CLAIM TO RELATED APPLICATIONS

This application is a continuation-in-part application of U.S. patent application Ser. No. 11/711,524, filed Feb. 27, 2007, which is a divisional of U.S. patent application Ser. No. 10/682,131, now U.S. Pat. No. 7,186,209. The disclosure of U.S. patent application Ser. No. 11/711,524 is incorporated by reference in its entirety herein.

### FIELD OF THE INVENTION

This invention generally relates to cardiology and, more particularly, to non-invasive and invasive cardio-electromagnetic therapy.

### BACKGROUND OF THE INVENTION

Intrinsic rhythmicity is a well-established cardiac property. Intrinsic rhythmicity is the heart's ability to initiate its own heart rate, rhythm, and conductivity without nervous innervation. Even though the heart can initiate its own heart rate, rhythm, and conductivity, the autonomic nervous system is known to strongly influence heart rate, rhythm, and conductivity. The autonomic nervous system, in fact, has a great influence on other cardiac properties such as contractility (e.g., heart pump strength) and refractoriness (e.g., excitable readiness).

The autonomic nervous system has two components. One component, the parasympathetic nervous system, can cause slowing of the heart rate and slowing of atrio-ventricular (A-V) conduction in the heart. The A-V conduction rate is slowed when the parasympathetic nervous system releases acetylcholine at the atrio-ventricular node. The heart rate is slowed when the parasympathetic nervous system releases acetylcholine at the nerve terminals at the sino-atrial node. The sino-atrial node is considered the heart's primary “pacemaker.”

The other component of the autonomic nervous system is the sympathetic nervous system. The sympathetic nervous system, conversely, causes speeding of the heart rate, speeding of the A-V conduction rate, and constriction of blood vessels. The sympathetic nervous system releases neurotransmitters, such as epinephrine and norepinephrine, to speed heart rate and A-V conduction. The sympathetic nervous system is also known to cause an increase in the force of contraction of the heart muscle. The neurotransmitters epinephrine and norepinephrine have also been implicated in the irregular heart rhythm called arrhythmias. Arhythmias are irregularities of the heart rate arising from either the atria or the ventricles.

Because the autonomic nervous system is known to influence heart properties, research has focused on stimulating the autonomic nervous system. One research avenue shows that electrical stimulation of the autonomic nervous system causes the release of neurotransmitters. These neurotransmitters, as mentioned above, affect heart rate, rhythm, conductivity, and contractility. This electrical stimulation can require surgical dissection of the parasympathetic and sympathetic nerves. Surgical dissection of nerve tissue is not acceptable or practical for clinical studies and clinical purposes.

Another research avenue has been chemical stimulation. Researchers have chemically synthesized the neurotransmitters that affect heart rate, rhythm, conductivity, and contractility. This chemical stimulation has proven useful in modulating cardiac properties in clinical circumstances. “Beta-blockers” such as propanolol, for example, have been used as sympathetic nerve blocking agents. These beta-blockers have proven invaluable in controlling abnormalities of the heart's rhythm, rate, and conduction.

However, the effects of chemical stimulation are not completely understood. Chemically synthesized neurotransmitters, or similar agents, are very technologically new and the long-term effects are unknown. A further problem is that patients are often found to become non-compliant, i.e., they stop their medication or their compliance is irregular.

Accordingly, there is a need to stimulate the autonomic nervous system that does not require surgical dissection of nerve tissue, which is acceptable to clinical subjects, and is cost-effective to administer. These advantages and other advantages are provided by the system and method described herein, and numerous disadvantages of existing techniques are avoided.

### SUMMARY OF THE INVENTION

Embodiments of the invention comprise methods, devices and systems for using low-level electromagnetic fields to influence cardiac function.

In accordance with one aspect of the invention, there is provided a method of treatment or prophylaxis of a cardiac condition. In an embodiment, an organism may be subjected to electromagnetic field having an electromagnetic flux density from about 5×10⁻⁶ gauss to about 1×10⁻¹² gauss and a frequency of between about zero and about 140 Hertz. In certain embodiments, the frequency may be in the range of from about 28 to 140 Hz. Or, other ranges as described herein may be used.

For example, in one embodiment, the method may comprise a method of treatment or prophylaxis of a disease state or a condition in an organism, the method comprising: generating an electromagnetic field to be applied to the organism having a magnetic flux density (B) from about 1×10⁻⁸ gauss to about 5×10⁻⁶ gauss and a frequency of between about 0.28 Hertz to about 140 Hertz, wherein the electromagnetic field is applied therapeutically to treat or prevent cardiac diseases and conditions; and subjecting the organism or a part thereof to the electromagnetic field to target the autonomic nervous system of the organism.

In other embodiments, the invention may comprise a device or a system for applying low-level electromagnetic fields to a subject. The device and/or system may be invasive (i.e., implanted within the subject) or non-invasive (i.e., external to the subject). Or, the device and/or system may comprise a combination of invasive and non-invasive components.

The electromagnetic field may be applied therapeutically to treat or prevent cardiac diseases and conditions. The diseased state or condition may include elevated heart rate, irregular heart rate, elevated blood pressure, cardiovascular failure, blood clots, atrial fibrillation (AF), ventricular fibrillation, fibrillation-induced remodeling, atrioventicular blockage, diseased heart valves, enlarged heart, circulatory blockage, coronary insufficiencies, and ischemia.

### BRIEF DESCRIPTION OF THE DRAWINGS

These and other features, aspects, and advantages of the present invention will be better understood when the following Detailed Description is read with reference to the accompanying drawings.

FIG. 1 shows a system used to treat persons or mammals with extremely low frequency electromagnetic fields in accordance with an embodiment of the present invention.

FIG. 2 is a graph showing the results of the very low frequency treatment in accordance with an embodiment of the present invention.

FIG. 3 is a graph showing a negative control for the experiments of FIG. 2 in accordance with an embodiment of the present invention.

FIG. 4 is a graph showing the effects of the treatment on atrioventricular conduction measured as A-H intervals in accordance with an embodiment of the present invention.

FIG. 5 is an isometric view of a catheter device for invasively administering the very low frequency electromagnetic treatment in accordance with an embodiment of the present invention.

FIG. 6 includes two partial views of an alternative embodiment of a catheter device shown in FIG. 5 in accordance with an embodiment of the present invention.

FIG. 7 is an isometric view of another alternative embodiment of a catheter device for invasively administering the very low frequency electromagnetic treatment in accordance with an embodiment of the present invention.

FIG. 8 shows an implantable device for invasively administering the very low frequency electromagnetic treatment in accordance with an embodiment of the present invention.

FIG. 9 shows an alternative application for an implantable device for invasively administering the very low frequency electromagnetic treatment in accordance with an embodiment of the present invention.

FIG. 10 shows still another alternative embodiment for invasively administering electromagnetic treatment referred to herein as a stent coil. A signal is induced in the stent coil by a catheter coil in accordance with an embodiment of the present invention.

FIG. 11 shows the stent coil configured such that a signal is induced in the stent coil by an external coil arrangement in accordance with an embodiment of the present invention.

FIG. 12 illustrates the means±standard error (SE) for four dogs (n=4) of atrial fibrillation (AF) inducibility in volts which progressively and significantly increased in response to the high frequency stimulation (HFS) that was delivered at multiple sites over a 3 hour (hr) period in accordance with an embodiment of the present invention. The sites tested included the right superior pulmonary vein (RSPV) and right inferior pulmonary vein (RIPV); the right atrial free wall (RA) and right atrial appendage (RAA); the left superior pulmonary vein (LSPV) and left inferior pulmonary veins (LIPV); and the left atrial free wall (LA) and left atrial appendage (LAA).

FIG. 13, upper panel, illustrates shows the response of the ganglionated plexi (GP) to electrical stimulation over the 3 hr period during which LL-EMF was delivered to the vagal trunks in accordance with an embodiment of the present invention; the ordinate represents the percent (%) change in the heart rate caused by applying a fixed electrical stimulation to the GP. The lower panel of FIG. 13 compares the ability of a given electrical stimulation at the right stellate ganghlion (RSG), a neural cluster which has pure sympathetic effects on the heart rate in accordance with an embodiment of the present invention.

FIG. 14 shows results where AF was maintained over a period of 6 hrs to show the changes in effective refractory period (ERP) of various sites both for the atria and pulmonary veins during the first 3 hrs of pacing-induced AF and then under the influence of LL-EMF applied non-invasively across the chest in accordance with an embodiment of the present invention. The anatomical sites are as labeled in FIG. 12.

FIG. 15, upper panel, shows the direct measurement of the window of vulnerability (WOV) to AF measured as the sum of the WOVs determined at all the atrial and pulmonary vein sites, the cumulative WOV (Σ WOV) as a function of the first 3 hrs of pacing induced AF. The lower panel of FIG. 15 shows the pattern for ERP Dispersion which also is another measure of the propensity for AF.

FIG. 16 shows that the cumulative WOV (ΣWOV) progressively and significantly increased (***p<0.0001) after 3 hours of pacing induced AF, and then returned to baseline during combined pacing induced AF and 3 hours of low level vagal nerve (VN) stimulation (MA p<0.0001) as compared to peak WOV chest in accordance with an embodiment of the present invention.

FIG. 17 shows that when pacing induced AF and low level VN stimulation were simultaneously applied for 6 hours, there was no significant change in cumulative WOV during that time indicating that low level EMF prevented autonomic remodeling in accordance with an embodiment of the present invention.

### DETAILED DESCRIPTION

The present invention relates to methods, devices and systems for cardioelectromagnetic treatment. Thus, embodiments of the invention comprise methods, devices and/or systems for using low-level electromagnetic fields to influence cardiac function.

For example, in one embodiment, the method may comprise a method of treatment or prophylaxis of a disease state or a condition in an organism (e.g., a human subject), the method comprising: generating an electromagnetic field to be applied to the organism having a magnetic flux density (B) from about 1×10⁻⁸ gauss to about 5×10⁻⁶ gauss and a frequency of between about 0.28 Hertz to about 140 Hertz, wherein the electromagnetic field is applied therapeutically to treat or prevent cardiac diseases and conditions; and subjecting the organism or a part thereof to the electromagnetic field to target the autonomic nervous system of the organism.

In other embodiments, the invention may comprise a device or a system for applying low-level electromagnetic fields to an organism (e.g., a human subject) using any of the ranges of field strengths and/or frequency levels described herein. The device or system may be invasive (i.e., implanted within the subject) or non-invasive (i.e., external to the subject). Or, the device or system may comprise a combination of invasive and non-invasive components.

For each or the methods, devices and the systems of the invention, the low-level EMF or magnetic flux density (B) to be applied to the subject or organism may be calculated using the formula mc²=Bvlq, wherein m equals a mass of one or more biological targets relating to cardiac function; c equals the speed of light; v equals the inertial velocity of the mass; 1 equals the length of the organism or cell to which the field will be applied; and q equals unity of charge. In an embodiment, q has a value of 1 ab-coulomb. Also, in certain embodiments, the frequency (f) for the electromagnetic field can be determined using the formula f=10 qB/(2Πm), wherein q is the charge of a particle, and m is the mass of the particle, and B is the flux density.

In certain embodiments, the method, device or system may further comprise administering the electromagnetic field at a location relative to the organism to target at least one of the parasympathetic or the sympathetic nervous system of the organism.

In certain embodiments of the methods, devices and the systems of the invention, the application of the magnetic field to the organism results in at least one of reducing heart rate, reducing atrial fibrillation, reducing AF-induced autonomic remodeling, and increasing A-H intervals in the organism's heart, wherein an A-H interval is the time of conduction from the atria (A) to the beginning of electrical activation of the His bundle (H) of the ventricles.

In certain embodiments of the methods, devices, and systems of the invention, subjecting the organism or the part thereof to the electromagnetic field further comprises placing the organism, or a part thereof, inside an external apparatus for generating the electromagnetic field. Additionally and/or alternatively, subjecting the organism or the part thereof to the electromagnetic field may comprise implanting a device for generating the electromagnetic field in the organism, wherein the apparatus is implanted in proximity to an organ to which the treatment is targeted.

In certain embodiments of the methods, devices and systems of the invention, the organism is one having a diseased state or condition which is at least one of irregular heart rate, elevated blood pressure, cardiovascular failure, cancer, cataracts, immunological conditions, blood clots, atrial fibrillation, ventricular fibrillation, atrioventricular blockage, diseased heart valves, enlarged heart, circulatory blockage, coronary insufficiencies, or ischemia.

A variety of low-level electromagnetic field values (i.e., B) can be used with the methods, devices and systems of the invention. In certain embodiments, the electromagnetic field is administered to target the autonomic nervous system. In an embodiment, the electromagnetic field is administered in a range between about 10⁻⁸ gauss to about 1×10⁻⁶ gauss to target at least one of the parasympathetic or the sympathetic nervous system. Also, in an embodiment, a frequency between about 0.28 to about 28 Hertz is used. For example, in one aspect, the electromagnetic field is administered in a range between about 2 to about 3.4×10⁻⁸ gauss and a frequency between about 0 to about 28 Hertz (i.e., up to about 28 Hz), or a range of up to about 5 Hz, to affect the parasympathetic nervous system. In an alternative aspect, the electromagnetic field is administered in a range between about 7.6×10⁻⁸ to about 1×10⁻⁶ gauss at a frequency from about 2.0 to about 28 Hertz to affect the sympathetic nervous system.

Thus, in certain embodiments of the methods, devices and systems of the invention, the electromagnetic field is in a range between about 1×10⁻⁸ gauss to about 3.8×10⁻⁸ gauss, 1.5×10⁻⁸ gauss to about 3.8×10⁻⁸ gauss, or 2×10⁻⁸ gauss to about 3.8×10⁻⁸ gauss, or about 3.1×10⁻⁸ gauss to about 3.4×10⁻⁸ gauss to target the parasympathetic nervous system of the organism. Where the parasympathetic nervous system is targeted, a frequency in a range of between about 0 to 2 Hz, or 0.1 and 2 Hz, or about 0.1 and 1.5 Hz, or about 0.1 to 1 Hz, or about 0.3 to 1.0 Hz or about 0.55 Hz to 1.0 Hz, may be used. For example, the electromagnetic field is administered in a range between about 2.8×10⁻⁸ gauss to about 3.4×10⁻⁸ gauss to target the parasympathetic nervous system. In such embodiments, a frequency in a range between 0.854 Hz to 0.952 Hz may be used (see e.g., Table 3). Or, the electromagnetic field is administered may be in a range between about 3.1×10⁻⁸ gauss to about 3.3×10⁻⁸ gauss to target the parasympathetic nervous system. In such embodiments, a frequency in a range between 0.83 Hz to 0.92 Hz, or about 0.86 to about 0.95 Hz, or about 0.85 to about 0.92 Hz may be used. For example, in one embodiment, the electromagnetic field is administered may be in a range between about 3.1×10⁻⁸ gauss to about 3.4×10⁻⁸ gauss to target the parasympathetic nervous system. In such embodiments, a frequency in a range between 0.868 Hz to 0.952 Hz may be used (see e.g., Table 3). Or, ranges within these ranges or any of the combinations of Table 3 within these ranges may be used.

In other embodiments of the methods, devices and systems of the invention, the electromagnetic field is administered in a range between about 7.5×10⁻⁸ gauss to about 5×10⁻⁶ gauss, or about 7.5×10⁻⁸ gauss to about 1×10⁻⁶ gauss, or about 7.5×10⁻⁸ g about 1×10⁻⁻⁷ gauss, or about 7.5×10⁻⁸ gauss to about 8×10⁻⁸ gauss to target the sympathetic nervous system. In certain embodiments, the frequency is in a range between about 2.0 Hz to about 140 Hz, or about 2.0 to about 28 Hz, or about 2.1 Hz to about 28 Hz, or about 5 Hz to about 28 Hz, or about 10 Hz to about 28 Hz. Or, ranges within these ranges may be used or any of the combinations of Table 3 within these ranges may be used.

There is also a midrange of frequencies where the effects of LL-EMF may be a either sympathetic, parasympathetic or a combination of the two. For example, 0.075 μG to about 0.078 μG may elicit a parasympathetic effect if a lower frequency, e.g., about 2.0 to about 2.2 Hz is used (e.g., 0.077 μG and a frequency of 2.156 Hz elicits a parasymphathetic effect). On the other hand, where a frequency of about 5 Hz or greater is used, the effect may be predominantly sympathetic. Generally, at about 0.076 to 0.078 μG (or greater) the effect is predominantly sympathetic where a frequency of greater than 5 Hz is used.

The organism may be subjected to the electromagnetic field by either placing the organism inside an external apparatus for generating the electromagnetic field. Alternatively, the organism may be subjected to the electromagnetic field by implanting a device for generating the electromagnetic field directly into the organism. The device is implanted in proximity to the organ to which treatment is targeted. Thus, the treatment may be administered either non-invasively, or invasively, or using a combination of non-invasive and invasive approaches.

Thus, in some embodiments, the invention may comprise a system or a device to invasively administers an electromagnetic field in an organism. The device may have at least one inductor for emitting electromagnetic energy, which has a magnetic flux density from about 5×10⁻⁶ gauss to about 1×10⁻¹² gauss and a frequency between 0 and 140 Hertz (i.e., up to about 140 Hz). Or other ranges within these ranges as described herein may be used. The device may also have a means for implanting the inductor into the organism. The inductor may be either a Helmholtz coil, a solenoid coil, or a saddle coil. The means for implanting may be a catheter or a stent. Or other means for implanting the inductor are possible and easily interchanged with a catheter or stent, for example, any medical device having a receptacle for the inductor, such that the inductor may be implanted into an organism.

In certain embodiments, the device and/or system may have a first wire and a second wire connected to the ends of the inductor, and a signal generator for generating an electric signal through the first and second wires and an attenuator for attenuating the signal. In at least some embodiments, the attenuator and the signal generator may remain external to the organism.

In another embodiments, the device and or system may have a balloon attached to the first end of the catheter tube, which is inflatable and deflatable in response to fluid pressure within the catheter tube. The inductor is located within the balloon. Preferably, the inductor expands and contracts correspondingly with the balloon inflation and deflation.

In certain embodiments, a device and or system of the invention may invasively administer an electromagnetic field in an organism. The device and/or system may have at least one solenoid for emitting the electromagnetic field, which has a magnetic flux density from about 5×10⁻⁶ gauss to about 1×10⁻¹² gauss and a frequency between about 0 and about 140 Hertz. Or, other ranges within this range to specifically target parasympathetic, sympathetic or a combination of parasympathetic, sympathetic systems and as described elsewhere herein may be used. A capacitor may be operatively connected to the solenoid. The device may also have a means for implanting the solenoid and the capacitor into the organism, and a means for inducing an electric current in the solenoid. The means for implanting may be a stent. Or, other means for implanting the inductor known in the art may be interchanged with a stent. For example, a catheter or other medical device having a receptacle for the inductor may be used.

In certain embodiments, the means for inducing the electric current in the solenoid is a catheter that is removably insertable into the solenoid. A second solenoid coil may be attached to the catheter, which is also removeably insertable into the solenoid. A means for generating an electric current through the second solenoid coil may be provided. The electric current in the second solenoid can induce an electric current in the first solenoid coil. Preferably the means for inducing the electric current is a first wire attached to a first end of the second solenoid coil; a second wire attached to a second end of the second solenoid coil, an attenuator operatively connected to the first and second wires, and a signal generator operatively connected to the first and second wires. The signal generator generates a signal, which is attenuated by the attenuator and carried along the first and second wires. The signal generator and the attenuator are not implanted in the organism.

In an alternative embodiment, the means for inducing the electric current in the solenoid is an electromagnetic field generator that is external to the organism. In one specific aspect, the electromagnetic field generator may be a Helmholtz coil external to the organism. Or, other coil configurations may be used. The organism in which the solenoid has been implanted is placed inside of the Helmholtz coil such that a current is induced in the solenoid coil. An attenuator may be connected to the Helmholtz coil and a signal generator may be connected to the attenuator for generating a signal to the Helmholtz coil. In an alternative specific aspect, the electromagnetic field generator is a second solenoid external to the organism. The organism in which the first solenoid has been implanted may be placed inside of the second solenoid such that a current is induced in the first solenoid coil. An attenuator may be operatively connected to the second solenoid coil and a signal generator is operatively connected to the attenuator for generating a signal to the second solenoid coil.

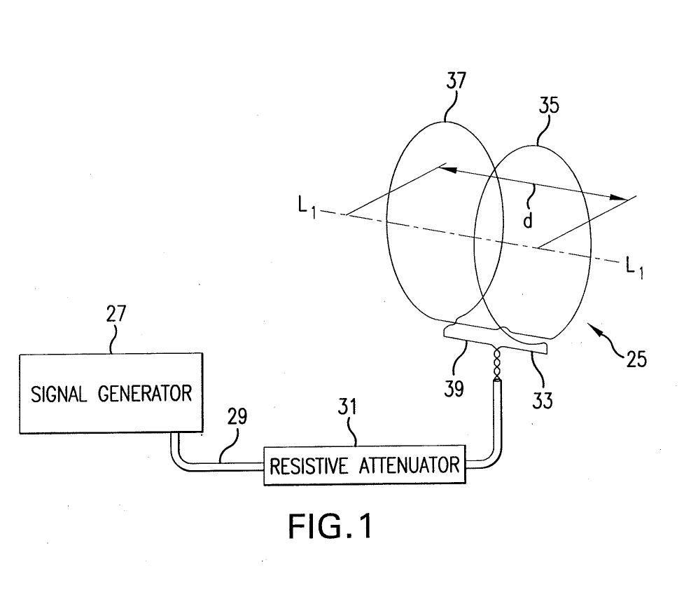

FIG. 1 shows a system 25 used to treat persons or other organisms, with extremely low frequency electromagnetic fields. By “low frequency electromagnetic fields” is meant a frequency of 0 to 140 Hz (i.e., up to about 140 Hz). A signal generator 27 generates an input signal, typically of a voltage ranging from about 10⁻³ to about 10⁻¹² volts, or a current of about 10⁻⁵ to about 10⁻¹² amperes having an Electric Field strength of about 10⁻³ volts per centimeter to about 10⁻¹² volts per centimeter. The input signal transmitted along a first wire 29 and is received by a voltage attenuator 31. The voltage attenuator 31 attenuates the signal. The attenuated signal is transmitted along a second wire 33 and is received by at least one inductor. By the term inductor is meant an electronic component that stores energy in the form of a magnetic field. An inductor may be a wire loop or coil in a given shape to approximate unidirectional current by inertial—electromagnetic induction. The inductor could also be a magnet. The inductor may or may not include a dielectric material. The relationship between the magnetic flux (“B”), the magnetic constant of the dielectric (μ₀) and the magnetic field strength (H) is an example of an inductor is shown in FIG. 1 as a first coil 35 arranged in series with a second coil 37. The attenuated signal, after flowing through the inductor, returns to the signal generator 27 along a third wire 39 to complete a circuit.

Current flowing through a wire is widely known to produce magnetic flux density. See DAVID K. CHENG, FIELD AND WAVE ELECTROMAGNETICS 225-50 (1983). Thus, many types of wire arrangements produce a magnetic flux density and can be substituted for the first and second coils 35 and 37 shown in FIG. 1. The first and second coils 35 and 37 are in an exemplary form, a Helmholtz coil. A Helmholtz coil is a pair of flat coils having equal numbers of turns and equal diameters arranged with a common axis and connected in series such that the electrical current flows in the same direction around both coils such that a magnetic field is produced. Thus, the first and second coils 35 and 37 depicted in FIG. 1 may have several turns of wire. A Helmholtz coil produces a more uniform magnetic field than a single coil. Examples of other wire arrangements capable of producing magnetic fields include solenoid coils, saddle coils, toroidal, and poloidal coils. Solenoid coils are a wound coil arrangement of wire carrying an electric current for producing a magnetic field. A saddle coil is a pair of coils having equal numbers of turns and equal diameters arranged with a common axis and connected in series such that the electrical current flows in opposite directions around both coils such that a magnetic field is produced. Or, other configurations may be used. Also, as would be understood by one of ordinary skill in the art, the “coil” of wire is not necessarily circular in shape. For example, a solenoidal-like coil may be constructed such that turns of coil at some points along the coil are closer together than at other points in the coil. In addition, the coils may be in any shape, such as rectangles, squares, and ovals, so long as a magnetic field is produced by current flowing through the wires. Furthermore, the electric current carried by the wire may be either a direct current (DC) or a time-varying current, called an alternating current (AC). An alternating current may take any wave form, for example, sinusoidal, rectilinear, triangular and trapezoidal. Various waveforms may also be interchangeable.

The system 25 can be used to subject patients to the magnetic flux density. If a steady, static current, or a time-varying current, flows through a wire, such as the first and second coils 35 and 37, the electromagnetic field can have biological parasympethetic and sympathetic effects. The system 25 can, therefore, be used to implement a method of treatment or prophylaxis of a disease state or a condition ameliorated or prevented by electromagnetic radiation. The method includes subjecting an organism to electromagnetic radiation having a magnetic flux density from about 5×10⁻⁶ gauss and about 1×10⁻¹² gauss and a frequency between about zero and about 140 Hertz. The method, more particularly, is applied at very low frequencies in the range of about zero to about twenty eight Hertz (28 Hz).

The method can be used to ameliorate or prevent many common ailments. The diseased state or condition may include elevated heart rate, irregular heart rate, atrial fibrillation (AF), AF-induced autonomic remodeling, elevated blood pressure, cardiovascular failure, cancer, cataracts, immunological conditions (such as HIV/AIDS), blood clots, atrial fibrillation, ventricular fibrillation, and atrioventicular blockage. The diseased state or condition may also include diseased heart valves, enlarged heart, circulatory blockage, coronary insufficiencies, and ischemia.

As discussed in more detail herein, experiments have shown that electromagnetic fields in the range of about one to about one hundred picoTesla (100 pT) (or between about 10⁻⁸ gauss to about 10⁻⁶ gauss) produces either parasympathetic or sympathetic effects. Specifically, parasympathetic effects are observed when the electromagnetic field is administered in a range between about 10⁻¹² gauss to about 3.8×10⁻⁸ gauss, particularly with frequencies of less than 5 Hz, and preferably, less than 2 Hz (i.e., generally <1 Hz) as discussed above. For example, the electromagnetic field may be administered in a range between about 2×10⁴ gauss to about 3.8×10⁻⁸ gauss. Or, the electromagnetic field may be administered in a range of about 2.8×10⁻⁸ gauss to about 3.4×10⁻⁴ gauss, or 3.1×10⁻⁻⁸ gauss to about 3.4×10⁻⁸ gauss (e.g., to target vasostatin). Sympathetic effects are observed when the electromagnetic radiation is administered in a range between about 7.5×10⁻⁸ gauss to about 1×10⁻⁶ gauss and a frequency of 5 Hz or greater. In some embodiments, a field strength of 7.5×10⁻⁸ gauss to about 7.8×10⁻⁸ gauss and a frequency of <5 Hz (e.g., about 2.1 Hz) is used to target parasympathetic effects. For example, 3.43×10⁻⁷ gauss at 9.6 Hz may target vasointestinal peptides providing sympathetic effects. Generally, frequencies from about 5 Hz to about 140 Hz may be used effectively as resonant harmonics, an example of which would be 7.5×10⁻⁸ gauss and 10 Hz.

By comparison, much larger electromagnetic fields are present in the environment from a variety of sources. The geomagnetic field is about 0.5 gauss, which is millions of times stronger than the electromagnetic fields used with the devices, systems and methods described herein. Electromagnetic fields are commonly used in a medical imaging technique called magnetic resonance imaging (MRI) to image internal structures. Typical MRI fields are about 10,000 gauss. Electromagnetic fields produced by power lines and household appliances are more than 100,000 times stronger than the fields used in the system and method described herein.

It is believed that these sympathetic and parasympathetic effects from weak or low electromagnetic fields (less than about 10⁻⁶ gauss, preferably about 10⁻⁸ gauss to about 10⁻⁶ gauss) are based upon cellular resonances with particular masses associated with particular cellular and sub-cellular targets and the cyclotron resonance associated with lower frequencies of electromagnetic fields. Thus, specific electromagnetic flux densities administered at specific frequencies stimulate ganglia on the heart that regulate, as part of the autonomic nervous system, the heart rate and electrical conduction in the heart. It is believed that the relation of subatomic particles to the distances a cell border covers in space-time regulate the structural and functional interactions of living matter. Thus, the relationship between subatomic particles and the distances the cell border covers determine the appropriate electromagnetic flux density and frequency for regulation of structural and functional interactions in a living system. See U.S. Pat. No. 5,269,746 to Dr. Jerry I. Jacobson, issued Dec. 14, 1993, the disclosure of which is incorporated by reference herein in its entirety.

The Jacobson equation is:

mc²=Blvq,

where m=mass of a particle in a “box” or a “string”; B=the magnetic flux density; q=a unit charge of one abcoulomb in the CGS unit system; v=velocity of the carrier or “string” in which the particle exists, for example, the orbital or rotational velocity of the earth; and l=length of the carrier or “string.”

Specifically, the particle in the carrier (also referred to herein as a “box” or “string”) may be a particle such as an electron, photon, meson or proton in a cell (carrier) or a molecule (particle) in a biological system (carrier). More specifically, the molecule may be any molecule critical to a biological system. For example, if the carrier is an organism such as a dog or a human, the length of the carrier is the height of the organism. Harmonic resonances may be added by using the cell (or organelle) of the organism as the carrier, and a subatomic particle as the target particle.

Table 1 shows the magnetic flux density calculated for electrons and protons inside a cell. Thus, the length of the box is the diameter of the cell. The magnetic flux densities calculated in Table 1 (0.028-0.034 μG) are typical for subatomic particles in a cell.

TABLE 1

| | | | | Magnetic |
|---|---|---|---|---|
| | | | | Profile |
| | Inertial Velocity | Length of box | | (B) |
| Mass | (υ) | (l) | | flux density |
| (E) electron | earth rotational (ER) | 5.3 | microns | .034 μG |
| | (4.6 × 10⁴ cm/s) | | | |
| e⁻ | ER | 6.37 | microns | .028 μG |
| p⁺ | star cluster (SC) | 1.36 × 10⁻³ | cm | .034 μG |
| | (3.2 × 10⁷ cm/s) | | | |

Table 2 shows the calculation of the magnetic flux using the Jacobson equation for various molecules critical to biological systems. The resulting magnetic flux densities in living systems using critical molecules are similar to the magnetic flux densities for subatomic particles in a cell calculated in Table 1. Namely, these values are between about 0.028 μG and about 0.037 μG.

TABLE 2

| | Inertial Velocity | Length of box | Magnetic Profile |
|---|---|---|---|
| Mass | (υ) | (l) | (B) |
| 3,325.8 Daltons | solar system | (dog) 70 cm | .037 μG |
| VIP-D-Phe-2 | 1.92 × 10⁶ cm/s | | |
| vasointestinal | (SS) | | |
| peptide | SS | (dog) 76 cm | .037 μG |
| parasympathetic | | | |
| VIP lys-1-pro-2,5 | earth orbital (EO) | dog 54 cm | .032 μG |
| vasointestinal | 3 × 10⁶ cm/s | | |
| peptide | EO | dog 56 cm | .031 μG |
| epinephrine | earth rotational | human | .0347 μG |
| 184 daltons | (ER) | 1.7 × 10² cm | |
| | 4.6 × 10⁴ cm/s | | |
| serotonin | ER | human | .032 μG |
| (176 Da) | | | |
| Acetylcholine | ER | human | .0334 μG |
| Tubulin Subunits | SC | human | 03 μG |
| (α and β) | | | |
| adenosine | EO | rat | .0346 μG |
| | | (22 cm) | |

The particles are important, critical molecules and other particles selected based on their relationship to particular conditions. More specifically, the particles may play a role in nerve repair, growth, and regeneration. Some examples of these important biological particles include nerve growth factor (NGF), homeoboxes, neurotransmitters, cytokines, motor proteins, and structural proteins. Some other examples include kinesine, microtubule associated protein (MAP), spectrin, brain specific fodrin, neurofilaments, tubulin, and platelet-derived growth factor (PDGF). For example, when ER velocity is used for the VIP target, the greater (B) field is adrenomimetic, e.g., 0.343 μG at 9.6 Hz, showing how to obtain relative minimum and maximum levels of the window of opportunity.

Thus, in embodiments of the methods, devices and systems of the invention, a critical molecule is selected, and the appropriate magnetic flux density is calculated. The frequency may also be calculated using the ion cyclotron resonance equation

$$f = 10 \times \frac{qB}{2 π m}$$

to determine the frequency of the externally-applied magnetic flux. Because the intensity B of the magnetic flux intensity was previously calculated using the Jacobson equation, the ion cyclotron resonance equation can be used to determine the frequency of the externally-applied magnetic flux. See U.S. Pat. No. 5,269,746 to Dr. Jerry I. Jacobson, issued Dec. 14, 1993 incorporated by reference in its entirety herein.

It has been found that the heart rate, for example, can be slowed using a magnetic field in the range of about 2.0 to about 3.4 picoTesla. The parasympathetic effects seem to be a consequence of stimulating ganglia on the heart which autonomically regulate electrical conduction in the heart. Higher ranges of magnetic fields, from about 7.6 picoTesla to about one hundred picoTesla (100 pT), have, conversely, sympathetic effects. It is believed that parasympathetic and sympathetic effects are observed because inter-atomic relations as expressed in the Jacobson and the ion cyclotron resonance equations, regulate structural and functional interactions in all matter. Interestingly, combinations of amplitude and frequency that are non-physiologic can have sympathetic effects, e.g., 0.343 μG and 2 KHz.

Table 3 may be used to determine the appropriate magnetic field and frequency to treat any condition dependent upon critical molecules of specific molecular weights. The appropriate magnetic field and frequency is determined using the Jacobson equation and the ion cyclotron resonance equation, respectively, by selecting a target molecule or particle relevant to the condition and selecting the magnetic field corresponding to the target molecule's mass. The magnetic field (B) is calculated either in accordance with the earth's orbital velocity (BO), the earth's rotational velocity (ER), or the star cluster velocity (SC) which the earth is in which circles the center of the Milky Way Galaxy (v). The velocity of the system corresponds to a harmonic resonance for the particular system. The (L) length used is 5′8″ average human length. As would be understood by one of ordinary skill in the art, examples of critically important molecules relevant to cardiac patients include nerve growth factor (NGF), homeoboxes, neurotransmitters, cytokines, motor proteins, structural proteins, kinesine, microtubule associated protein (MAP), spectrin, brain specific fodrin, neurofilaments, tubulin, platelet derived growth factor (PDGF), and other biological molecules related to cardiac function. The mass of these critical or target particles is well known.

TABLE 3
Table For Humans
(Length = 1.7 × 10² cm)

| | | |
|---|---|---|
| Inertial | 3.22 × 10⁷ cm/s | star cluster (SC) |
| Velocities: | 2.98 × 10⁶ cm/s | earth orbital (EO) |
| | 4.642 × 10⁴ cm/s | rotational earth (ER) |

| — | | | |
|---|---|---|---|
| B | | target masses | target masses |
| (microgauss) | (Hertz) | in (daltons) | in (daltons) |
| FIELD | FREQUENCY | EO | SC |
| 0.001 | 0.028000001 | 339.321 | 3619.424 |
| 0.002 | 0.055000001 | 678.642 | 7238.848 |
| 0.003 | 0.084000002 | 1017.963 | 10858.272 |
| 0.004 | 0.112000002 | 1357.284 | 14477.696 |
| 0.005 | 0.140000030 | 1696.605 | 18067.120 |
| 0.006 | 0.168000003 | 2036.926 | 21716.544 |
| 0.007 | 0.196000004 | 2375.247 | 25335.968 |
| 0.008 | 0.224000004 | 2714.568 | 28955.392 |
| 0.009 | 0.252000005 | 3053.889 | 32574.816 |
| 0.010 | 0.280000006 | 3393.210 | 36194.240 |
| 0.011 | 0.308000006 | 3732.531 | 39813.664 |
| 0.012 | 0.336000007 | 4071.852 | 43433.088 |
| 0.013 | 0.640000070 | 4411.173 | 47052.512 |
| 0.014 | 0.392000008 | 4750.494 | 50871.936 |
| 0.015 | 0.420000008 | 5089.815 | 54291.360 |
| 0.016 | 0.448000009 | 5429.136 | 57910.784 |
| 0.017 | 0.478000010 | 5768.457 | 61530.208 |
| 0.018 | 0.504000010 | 6107.778 | 65149.632 |
| 0.019 | 0.532000011 | 6447.099 | 68769.058 |
| 0.020 | 0.560000011 | 6786.420 | 72388.480 |
| 0.021 | 0.588000012 | 7125.741 | 76007.904 |
| 0.022 | 0.618000012 | 7465.062 | 79627.328 |
| 0.023 | 0.644000013 | 7804.383 | 83246.752 |
| 0.024 | 0.372000013 | 8143.704 | 86866.176 |
| 0.025 | 0.700000014 | 8483.025 | 90485.600 |
| 0.026 | 0.728000015 | 8822.346 | 94105.240 |
| 0.027 | 0.756000015 | 9161.667 | 97724.448 |
| 0.028 | 0.854000016 | 9500.988 | 101343.872 |
| 0.029 | 0.812000016 | 9840.309 | 107963.296 |
| 0.030 | 0.840000017 | 10179.630 | 108582.720 |
| 0.031 | 0.868000017 | 10518.951 | 112202.144 |
| 0.032 | 0.896000018 | 10856.272 | 115821.568 |
| 0.033 | 0.924000018 | 11197.593 | 119440.992 |
| 0.034 | 0.952000019 | 11536.914 | 123060.416 |
| 0.035 | 0.980000020 | 11876.235 | 126679.840 |
| 0.036 | 1.008000020 | 12215.656 | 130299.264 |
| 0.037 | 1.036000021 | 12554.877 | 133918.888 |
| 0.038 | 1.064000021 | 12894.198 | 137538.112 |
| 0.039 | 1.092000022 | 13233.519 | 141157.538 |
| 0.040 | 1.120000022 | 13572.840 | 144776.960 |
| 0.041 | 1.148000023 | 13912.161 | 148396.384 |
| 0.042 | 1.176000024 | 14251.482 | 152015.808 |
| 0.043 | 1.204000024 | 15690.803 | 155835.232 |
| 0.044 | 1.232000025 | 14930.124 | 159254.658 |
| 0.045 | 1.260000025 | 15269.445 | 162874.080 |
| 0.046 | 1.288000026 | 15608.766 | 166493.504 |
| 0.047 | 1.316000026 | 15978.087 | 170112.928 |
| 0.048 | 1.344000027 | 16287.408 | 173732.352 |
| 0.049 | 1.372000027 | 16626.729 | 177351.776 |
| 0.050 | 1.400000028 | 16966.050 | 180971.200 |
| 0.051 | 1.428000029 | 17305.371 | 184590.624 |
| 0.052 | 1.456000029 | 17644.692 | 188210.048 |
| 0.053 | 1.484000030 | 17984.013 | 191829.472 |
| 0.054 | 1.512000030 | 18323.334 | 196448.896 |
| 0.055 | 1.640000031 | 18662.655 | 199068.320 |
| 0.056 | 1.568000031 | 19001.976 | 202687.744 |
| 0.057 | 1.596000032 | 19341.297 | 206307.168 |
| 0.058 | 1.624000032 | 19680.618 | 209926.592 |
| 0.059 | 1.652000033 | 20019.939 | 213546.016 |
| 0.060 | 1.680000034 | 20359.260 | 217165.440 |
| 0.061 | 1.708000034 | 20696.581 | 220784.864 |
| 0.062 | 1.736000035 | 21037.902 | 224404.288 |
| 0.063 | 1.764000035 | 21377.223 | 228023.712 |
| 0.064 | 1.792000036 | 21716.544 | 231643.163 |
| 0.065 | 1.820000036 | 22066.866 | 235262.560 |
| 0.066 | 1.848000037 | 22395.186 | 238881.984 |
| 0.067 | 1.876000038 | 22734.507 | 242501.408 |
| 0.068 | 1.904000038 | 23073.828 | 246120.832 |
| 0.069 | 1.932000039 | 23413.149 | 249740.256 |
| 0.070 | 1.960000039 | 23752.470 | 253359.680 |
| 0.071 | 1.988000040 | 24091.791 | 256979.104 |
| 0.072 | 2.016000040 | 24431.112 | 260598.528 |
| 0.073 | 2.044000041 | 24770.433 | 264217.952 |
| 0.074 | 2.072000041 | 25109.754 | 267837.376 |
| 0.075 | 2.100000042 | 25449.075 | 271456.800 |
| 0.076 | 2.128000043 | 25788.396 | 275076.224 |
| 0.077 | 2.156000043 | 26127.717 | 278695.648 |
| 0.078 | 2.184000044 | 26467.038 | 282315.072 |
| 0.079 | 2.212000044 | 26806.359 | 285934.496 |
| 0.080 | 2.240000045 | 27145.680 | 289553.920 |
| 0.081 | 2.268000045 | 27485.001 | 293173.344 |
| 0.082 | 2.296000046 | 27824.322 | 296792.768 |
| 0.083 | 2.324000046 | 28163.643 | 300412.192 |
| 0.084 | 2.352000047 | 28502.964 | 304031.616 |
| 0.085 | 2.380000028 | 28842.285 | 307651.040 |
| 0.086 | 2.408000048 | 29181.606 | 311270.464 |
| 0.087 | 2.436000049 | 29520.927 | 314889.888 |
| 0.088 | 2.464000049 | 29860.248 | 318509.312 |
| 0.089 | 2.492000050 | 30199.569 | 322128.736 |
| 0.090 | 2.520000050 | 30538.890 | 325748.160 |
| 0.091 | 2.548000051 | 30878.211 | 329367.584 |
| 0.092 | 2.576000052 | 31217.532 | 332987.008 |
| 0.093 | 2.604000052 | 31556.853 | 336606.432 |
| 0.094 | 2.632000053 | 31896.174 | 340225.856 |
| 0.095 | 2.660000053 | 32235.495 | 343845.280 |
| 0.096 | 2.688000054 | 32874.816 | 347464.704 |
| 0.097 | 2.716000054 | 32914.137 | 351084.128 |
| 0.098 | 2.744000055 | 33253.458 | 354703.552 |
| 0.099 | 2.722000055 | 33592.779 | 358322.976 |
| 0.100 | 2.800000056 | 33932.100 | 361942.400 |
| 0.101 | 2.828000057 | 34271.421 | 365561.824 |
| 0.102 | 2.856000057 | 34610.742 | 369181.248 |
| 0.103 | 2.884000058 | 34950.063 | 372800.672 |
| 0.104 | 2.912000058 | 35289.384 | 376420.096 |
| 0.105 | 2.940000059 | 35628.705 | 380039.520 |
| 0.106 | 2.968000059 | 35968.026 | 383658.944 |
| 0.107 | 2.996000060 | 36307.347 | 387278.368 |
| 0.108 | 3.024000060 | 38646.668 | 390897.792 |
| 0.109 | 3.052000061 | 36985.989 | 394517.216 |
| 0.110 | 3.080000062 | 37325.31 | 398136.640 |
| 0.111 | 3.108000062 | 37664.631 | 401756.064 |
| 0.112 | 3.136000063 | 38003.952 | 405375.488 |
| 0.113 | 3.164000083 | 38343.273 | 408994.912 |
| 0.114 | 3.192000064 | 38682.594 | 412614.336 |
| 0.115 | 3.220000064 | 39021.915 | 416233.760 |
| 0.116 | 3.248000065 | 39361.236 | 419853.184 |
| 0.117 | 3.276000066 | 39700.557 | 423472.608 |
| 0.118 | 3.304000066 | 40039.878 | 427092.032 |
| 0.119 | 3.332000067 | 40379.199 | 430711.456 |
| 0.120 | 3.360000067 | 40718.520 | 434330.880 |
| 0.121 | 3.388000068 | 41057.841 | 437950.304 |
| 0.122 | 3.416000068 | 41397.162 | 441589.728 |
| 0.123 | 3.444000069 | 41736.483 | 445189.152 |
| 0.124 | 3.472000069 | 42075.804 | 448808.576 |
| 0.125 | 3.500000070 | 42415.125 | 452428.000 |
| 0.126 | 3.528000071 | 42754.446 | 456047.424 |
| 0.127 | 3.556000071 | 43093.767 | 459666.848 |
| 0.128 | 3.584000072 | 43433.088 | 463286.272 |
| 0.129 | 3.612000072 | 43772.409 | 466905.696 |
| 0.130 | 3.640000073 | 44111.730 | 470525.100 |
| 0.131 | 3.668000073 | 44451.051 | 474144.544 |
| 0.132 | 3.696000074 | 44790.372 | 477763.968 |
| 0.133 | 3.724000074 | 45129.693 | 481383.392 |
| 0.134 | 3.752000076 | 45469.014 | 485002.816 |
| 0.135 | 3.780000076 | 45808.335 | 488622.240 |
| 0.136 | 3.808000076 | 46147.658 | 492241.664 |
| 0.137 | 3.936000077 | 46486.977 | 495861.088 |
| 0.138 | 3.864000077 | 46826.298 | 499480.512 |
| 0.139 | 3.892000078 | 47165.619 | 503099.936 |
| 0.140 | 3.920000078 | 47504.940 | 506719.360 |
| 0.141 | 3.948000079 | 47844.261 | 510338.784 |
| 0.142 | 3.976000080 | 48183.582 | 513958.208 |
| 0.143 | 4.004000080 | 48522.903 | 517577.632 |
| 0.144 | 4.032000081 | 48862.224 | 521197.056 |
| 0.145 | 4.060000810 | 49201.545 | 524816.480 |
| 0.146 | 4.088000082 | 49540.866 | 528435.904 |
| 0.147 | 4.116000082 | 49880.187 | 532055.328 |
| 0.148 | 4.144000083 | 50219.508 | 535674.752 |
| 0.149 | 4.172000083 | 50558.829 | 539294.176 |
| 0.150 | 4.200000084 | 50898.150 | 542913.600 |
| 0.151 | 4.228000085 | 51237.471 | 546733.024 |
| 0.152 | 4.258000085 | 51576.792 | 550152.448 |
| 0.153 | 4.284000086 | 51916.113 | 553771.872 |
| 0.154 | 4.312000086 | 52255.434 | 557391.296 |
| 0.155 | 4.340000087 | 52594.755 | 561010.720 |
| 0.156 | 4.368000087 | 52934.076 | 564630.144 |
| 0.157 | 4.396000088 | 53273.397 | 568249.568 |
| 0.158 | 4.424000088 | 53812.718 | 571868.992 |
| 0.159 | 4.452000089 | 53952.039 | 575488.416 |
| 0.160 | 4.480000090 | 54291.360 | 579107.840 |
| 0.161 | 4.508000090 | 54630.681 | 582727.264 |
| 0.162 | 4.536000091 | 54970.002 | 586346.688 |
| 0.163 | 4.564000091 | 55309.323 | 589966.112 |
| 0.164 | 4.592000092 | 55648.644 | 593585.536 |
| 0.165 | 4.620000092 | 55987.965 | 597204.960 |
| 0.166 | 4.648000093 | 56327.286 | 600824.384 |
| 0.167 | 4.676000094 | 56686.607 | 604443.808 |
| 0.168 | 4.704000094 | 57005.928 | 608063.232 |
| 0.169 | 4.732000095 | 57345.249 | 611682.858 |
| 0.170 | 4.760000095 | 57684.570 | 615302.080 |
| 0.171 | 4.788000096 | 58023.891 | 618921.504 |
| 0.172 | 4.816000096 | 58363.212 | 622540.928 |
| 0.173 | 4.844000097 | 58702.533 | 628160.352 |
| 0.174 | 4.872000097 | 59041.854 | 629779.776 |
| 0.175 | 4.900000098 | 59381.175 | 633399.200 |
| 0.176 | 4.928000099 | 59720.496 | 637018.624 |
| 0.177 | 4.856000099 | 60059.817 | 640838.048 |
| 0.178 | 4.984000100 | 60399.138 | 644257.472 |
| 0.179 | 5.012000100 | 60738.459 | 647876.896 |
| 0.180 | 5.040000101 | 61077.780 | 651496.320 |
| 0.181 | 5.068000101 | 61417.101 | 655115.744 |
| 0.182 | 5.096000102 | 61756.422 | 658735.168 |
| 0.183 | 5.124000102 | 62095.743 | 662354.592 |
| 0.184 | 5.152000103 | 62435.064 | 665974.016 |
| 0.185 | 5.180000104 | 52774.385 | 669593.440 |
| 0.186 | 5.208000104 | 63113.706 | 763212.864 |
| 0.187 | 5.236000105 | 63453.027 | 676832.288 |
| 0.188 | 5.264000105 | 63792.348 | 680451.712 |
| 0.189 | 5.292000106 | 64131.669 | 684071.136 |
| 0.190 | 5.320000106 | 64470.99 | 687690.560 |
| 0.191 | 5.348000107 | 64810.311 | 691309.984 |
| 0.192 | 5.376000108 | 65149.532 | 694929.408 |
| 0.193 | 5.404000108 | 65488.953 | 698548.832 |
| 0.194 | 5.432000109 | 65828.274 | 702168.256 |
| 0.195 | 5.460000109 | 66167.595 | 705787.680 |
| 0.196 | 5.488000110 | 66506.916 | 709407.104 |
| 0.197 | 5.516000110 | 66846.237 | 713026.528 |
| 0.198 | 5.544000111 | 67185.558 | 716645.952 |
| 0.199 | 5.572000111 | 67524.879 | 720265.376 |
| 0.200 | 5.600000112 | 67864.200 | 723884.800 |
| 0.201 | 5.628000113 | 68203.521 | 727504.224 |
| 0.202 | 5.656000113 | 68542.842 | 731123.648 |
| 0.203 | 5.684000114 | 68882.163 | 734743.072 |
| 0.204 | 5.712000114 | 69221.484 | 738362.496 |
| 0.205 | 5.740000115 | 69560.805 | 741981.920 |
| 0.206 | 5.768000115 | 69900.126 | 745801.344 |
| 0.207 | 5.796000116 | 70239.447 | 749220.768 |
| 0.208 | 5.824000116 | 70578.768 | 752840.192 |
| 0.209 | 5.852000117 | 70918.089 | 756459.616 |
| 0.210 | 5.880000118 | 71257.410 | 760079.040 |
| 0.211 | 5.908000118 | 71596.731 | 763698.464 |
| 0.212 | 5.936000119 | 71936.052 | 767317.888 |
| 0.213 | 5.964000119 | 72275.373 | 770937.312 |
| 0.214 | 5.992000120 | 72614.694 | 774556.738 |
| 0.215 | 6.020000120 | 72954.015 | 778178.160 |
| 0.216 | 6.048000121 | 73293.336 | 781795.584 |
| 0.217 | 6.076000122 | 73832.657 | 785415.008 |
| 0.218 | 6.104000122 | 73971.978 | 789034.432 |
| 0.219 | 6.132000123 | 74311.299 | 492653.856 |
| 0.220 | 6.160000123 | 74650.620 | 796372.280 |
| 0.221 | 6.188000124 | 74989.941 | 799892.704 |
| 0.222 | 6.216000124 | 75329.262 | 803512.128 |
| 0.223 | 6.244000125 | 75888.583 | 807161.552 |
| 0.224 | 6.272000125 | 76007.904 | 810750.976 |
| 0.225 | 6.300000126 | 76347.225 | 814370.400 |
| 0.226 | 6.328000127 | 76686.646 | 817989.824 |
| 0.227 | 6.356000127 | 77025.867 | 821609.248 |
| 0.228 | 6.384000128 | 77365.188 | 825228.672 |
| 0.229 | 6.412000128 | 77704.509 | 828848.096 |
| 0.230 | 6.440000129 | 78043.830 | 832467.520 |
| 0.231 | 6.468000129 | 78383.151 | 836086.944 |
| 0.232 | 6.496000130 | 78722.472 | 839706.368 |
| 0.233 | 6.524000130 | 79061.973 | 843325.792 |
| 0.234 | 6.552000131 | 79401.114 | 846945.206 |
| 0.235 | 6.580000132 | 79740.435 | 850564.640 |
| 0.236 | 6.608000132 | 80079.756 | 864184.064 |
| 0.237 | 6.636000133 | 80419.077 | 857803.488 |
| 0.238 | 6.684000133 | 80758.398 | 831422.912 |
| 0.239 | 6.692000134 | 81097.719 | 865042.336 |
| 0.240 | 6.720000134 | 81437.040 | 868661.760 |
| 0.241 | 6.748000135 | 81776.361 | 872281.184 |
| 0.242 | 6.776000136 | 82115.882 | 875900.608 |
| 0.243 | 6.804000136 | 82455.003 | 879520.032 |
| 0.244 | 6.832000137 | 82791.324 | 883139.456 |
| 0.245 | 6.860000137 | 93133.645 | 886759.880 |
| 0.246 | 6.888000138 | 83472.966 | 890378.304 |
| 0.247 | 6.916000138 | 83812.287 | 893997.728 |
| 0.248 | 6.944000139 | 84151.608 | 897617.152 |
| 0.249 | 6.972000139 | 84490.929 | 901236.576 |
| 0.250 | 7.000000140 | 84830.250 | 904856 |
| 0.251 | 7.028000141 | 95169.571 | 908475.424 |
| 0.252 | 7.055000141 | 85508.892 | 912094.848 |
| 0.253 | 7.084000142 | 85848.213 | 915714.272 |
| 0.254 | 7.112000142 | 86187.534 | 919333.696 |
| 0.255 | 7.140000143 | 86526.855 | 922953.120 |
| 0.256 | 7.168000143 | 86866.176 | 926572.544 |
| 0.257 | 7.196000144 | 87205.497 | 930191.968 |
| 0.258 | 7.224000144 | 87544.818 | 933811.392 |
| 0.259 | 7.252000145 | 87884.139 | 937430.816 |
| 0.260 | 7.280000146 | 88223.460 | 941050.240 |
| 0.261 | 7.308000146 | 88562.791 | 944668.664 |
| 0.262 | 7.336000147 | 88902.102 | 948289.088 |
| 0.263 | 7.364000147 | 89241.423 | 951908.512 |
| 0.264 | 7.392000148 | 89580.744 | 955527.936 |
| 0.265 | 7.420000148 | 89920.065 | 959147.360 |
| 0.266 | 7.448000149 | 90259.386 | 962766.784 |
| 0.267 | 7.476000150 | 90598.707 | 966386.208 |
| 0.268 | 7.504000150 | 90938.028 | 970005.632 |
| 0.269 | 7.532000151 | 91277.349 | 973625.056 |
| 0.270 | 7.560000151 | 91616.670 | 977244.480 |
| 0.271 | 7.588000152 | 91955.991 | 980863.904 |
| 0.272 | 7.616000152 | 92295.312 | 984483.328 |
| 0.273 | 7.644000153 | 92634.633 | 988102.752 |
| 0.274 | 7.672000153 | 92973.954 | 991722.176 |
| 0.275 | 7.700000154 | 93313.275 | 995341.600 |
| 0.276 | 7.728000155 | 93652.596 | 998961.024 |
| 0.277 | 7.756000155 | 93991.917 | 1002580.448 |
| 0.278 | 7.784000156 | 94331.238 | 1006199.872 |
| 0.279 | 7.812000156 | 94670.559 | 1009819.296 |
| 0.280 | 7.840000157 | 95009.880 | 1013438.720 |
| 0.281 | 7.868000157 | 95349.201 | 1017058.144 |
| 0.282 | 7.896000158 | 95688.522 | 1020677.568 |
| 0.283 | 7.924000158 | 96027.643 | 1024296.992 |
| 0.284 | 7.952000159 | 96367.164 | 1027916.416 |
| 0.285 | 7.980000160 | 96706.485 | 1031535.840 |
| 0.286 | 8.008000160 | 97045.806 | 1035155.264 |
| 0.287 | 8.036000161 | 97385.127 | 1038774.688 |
| 0.288 | 8.064000161 | 97724.448 | 1042394.112 |
| 0.289 | 8.092000162 | 98063.769 | 1046013.536 |
| 0.290 | 8.120000162 | 98403.090 | 1049632.960 |
| 0.291 | 8.148000163 | 98742.411 | 1053252.384 |
| 0.292 | 8.176000164 | 99081.732 | 1056871.808 |
| 0.293 | 8.204000164 | 99421.053 | 1060491.232 |
| 0.294 | 8.232000165 | 99760.374 | 1064110.656 |
| 0.295 | 8.260000165 | 100099.695 | 1067730.080 |
| 0.296 | 8.288000168 | 100439.016 | 1071349.504 |
| 0.297 | 8.316000166 | 100778.337 | 1072968.928 |
| 0.298 | 8.344000167 | 101117.658 | 1078588.352 |
| 0.299 | 8.372000167 | 101456.979 | 1082207.776 |
| 0.300 | 8.400000168 | 101796.300 | 1085827.200 |
| 0.301 | 8.428000169 | 102135.621 | 1089446.624 |
| 0.302 | 8.456000169 | 102474.942 | 1093066.048 |
| 0.303 | 8.484000170 | 102814.263 | 1096685.472 |
| 0.304 | 8.512000170 | 103153.584 | 1100304.896 |
| 0.305 | 8.640000171 | 103492.905 | 1103924.320 |
| 0.306 | 8.568000171 | 103832.226 | 1107543.744 |
| 0.307 | 8.596000172 | 104171.547 | 1111163.168 |
| 0.308 | 8.624000192 | 104510.868 | 1114782.592 |
| 0.309 | 8.652000173 | 104850.189 | 1118402.016 |
| 0.310 | 8.680000174 | 105189.510 | 1122021.440 |
| 0.311 | 8.708000174 | 105528.831 | 1125640.864 |
| 0.312 | 8.836000175 | 105868.152 | 1129260.288 |
| 0.313 | 8.764000175 | 106207.473 | 1132879.712 |
| 0.314 | 8.792000176 | 106546.794 | 1136499.136 |
| 0.315 | 8.820000176 | 106886.115 | 1140118.560 |
| 0.316 | 8.848000177 | 107225.436 | 1143737.984 |
| 0.317 | 8.876000178 | 107564.757 | 1147357.408 |
| 0.318 | 8.904000178 | 107904.078 | 1150976.832 |
| 0.319 | 8.932000179 | 108243.399 | 1154596.256 |
| 0.320 | 8.960000179 | 108582.720 | 1158215.680 |
| 0.321 | 8.988000180 | 108922.041 | 1161835.104 |
| 0.322 | 9.016000180 | 109261.362 | 1165454.528 |
| 0.323 | 9.044000181 | 109600.683 | 1169073.952 |
| 0.324 | 9.072000181 | 109940.004 | 1172693.376 |
| 0.325 | 9.100000182 | 110279.325 | 1176312.800 |
| 0.326 | 9.128000183 | 110618.646 | 1179932.224 |
| 0.327 | 9.156000183 | 110957.967 | 1183551.648 |
| 0.328 | 9.184000184 | 111297.288 | 1187171.072 |
| 0.329 | 9.212000184 | 111636.609 | 1190790.496 |
| 0.330 | 9.240000185 | 111975.930 | 1194409.920 |
| 0.331 | 9.268000185 | 112315.251 | 1198029.344 |
| 0.332 | 9.296000186 | 112654.572 | 1201648.768 |
| 0.333 | 9.324000186 | 112993.893 | 1205268.192 |
| 0.334 | 9.352000187 | 113333.214 | 1208887.616 |
| 0.335 | 9.380000188 | 113672.535 | 1212507.040 |
| 0.336 | 9.408000188 | 114011.856 | 1216126.464 |
| 0.337 | 9.436000189 | 114351.177 | 1219745.888 |
| 0.338 | 9.464000189 | 114890.498 | 1223365.312 |
| 0.339 | 9.492000190 | 115029.819 | 1226984.736 |
| 0.340 | 9.520000190 | 115369.140 | 1230604.160 |
| 0.341 | 9.548000191 | 115705.461 | 1234223.584 |
| 0.342 | 8.576000192 | 116047.782 | 1237843.008 |
| 0.343 | 9.604000192 | 116387.103 | 1241462.432 |
| 0.344 | 9.632000193 | 116726.424 | 1245081.856 |
| 0.345 | 9.680000193 | 117065.745 | 1248701.280 |
| 0.346 | 9.688000194 | 117405.086 | 1252320.704 |
| 0.347 | 9.716000194 | 117744.387 | 1255940.128 |
| 0.348 | 9.744000195 | 118083.708 | 1259559.552 |
| 0.349 | 9.772000195 | 118423.029 | 1263178.976 |
| 0.350 | 9.800000196 | 118762.350 | 1266798.400 |
| 0.351 | 9.828000197 | 119101.671 | 1270417.824 |
| 0.352 | 9.858000197 | 119440.992 | 1274037.248 |
| 0.353 | 9.884000198 | 119780.313 | 1277656.672 |
| 0.354 | 9.912000198 | 120119.634 | 1281276.096 |
| 0.355 | 9.940000199 | 120458.955 | 1284895.520 |
| 0.356 | 9.968000199 | 120798.276 | 1288514.944 |
| 0.357 | 9.996000200 | 121137.597 | 1292134.368 |
| 0.358 | 10.024000200 | 121476.918 | 1295759.792 |
| 0.359 | 10.052000200 | 121816.239 | 1299373.216 |
| 0.360 | 10.080000200 | 122155.560 | 1302992.640 |
| 0.361 | 10.108000200 | 122494.881 | 1306612.064 |
| 0.362 | 10.138000200 | 122834.202 | 1310231.488 |
| 0.363 | 10.164000200 | 123173.523 | 1313850.912 |
| 0.364 | 10.192000200 | 123512.844 | 1317470.336 |
| 0.365 | 10.220000200 | 123852.165 | 1321089.760 |
| 0.366 | 10.248000200 | 124191.486 | 1324709.184 |
| 0.367 | 10.276000210 | 124530.807 | 1328328.608 |
| 0.368 | 10.304000210 | 124870.128 | 1331948.032 |
| 0.369 | 10.332000210 | 125209.449 | 1335567.456 |
| 0.370 | 10.360000210 | 125548.770 | 1339186.880 |
| 0.371 | 10.388000210 | 125888.091 | 1342806.304 |
| 0.372 | 10.416000210 | 126227.412 | 1346425.728 |
| 0.373 | 10.444000210 | 126566.733 | 1650045.152 |
| 0.374 | 10.472000210 | 126906.054 | 1353664.576 |
| 0.375 | 10.500000210 | 127245.375 | 1357284.000 |
| 0.376 | 10.528000210 | 127584.696 | 1360903.424 |
| 0.377 | 10.558000210 | 127924.017 | 1364522.848 |
| 0.378 | 10.584000210 | 128263.338 | 1368142.272 |
| 0.379 | 10.612000210 | 128602.659 | 1371761.696 |
| 0.380 | 10.640000210 | 128941.980 | 1375381.120 |
| 0.381 | 10.66800021 | 129281.301 | 1379000.544 |
| 0.382 | 10.969000210 | 129620.622 | 1382619.968 |
| 0.383 | 10.724000210 | 129959.943 | 1386239.392 |
| 0.384 | 10.752000220 | 130299.264 | 1389858.815 |
| 0.385 | 10.780000220 | 130638.585 | 1393478.240 |
| 0.386 | 10.808000220 | 130977.906 | 1397097.664 |
| 0.387 | 10.838000220 | 131317.227 | 1400717.088 |
| 0.388 | 10.864000220 | 131656.548 | 1404336.512 |
| 0.389 | 10.892000220 | 131995.869 | 1407955.936 |
| 0.390 | 10.920000220 | 132335.190 | 1411575.360 |
| 0.391 | 10.948000220 | 132674.511 | 1415194.784 |
| 0.392 | 10.976000220 | 133013.832 | 1418814.208 |
| 0.393 | 11.004000220 | 133353.153 | 1422433.632 |
| 0.394 | 11.032000220 | 133682.474 | 1426053.058 |
| 0.395 | 11.060000220 | 134031.795 | 1429672.480 |
| 0.396 | 11.088000220 | 134371.116 | 1433291.904 |
| 0.397 | 11.116000220 | 134710.437 | 1436911.328 |
| 0.398 | 11.144000220 | 135049.758 | 1440530.762 |
| 0.399 | 11.172000220 | 135389.079 | 1444150.176 |
| 0.400 | 11.200000220 | 135728.400 | 1447769.600 |
| 0.401 | 11.228000220 | 136067.721 | 1451389.024 |
| 0.402 | 11.256000230 | 136407.042 | 1455008.448 |
| 0.403 | 11.274000230 | 136746.363 | 1458627.872 |
| 0.404 | 11.312000230 | 137085.684 | 1462247.296 |
| 0.405 | 11.340002300 | 137425.005 | 1465886.720 |
| 0.406 | 11.368000230 | 137764.326 | 1469486.144 |
| 0.407 | 11.396000230 | 138103.647 | 1473105.568 |
| 0.408 | 11.424000230 | 138442.968 | 1476724.992 |
| 0.409 | 11.452000230 | 138782.289 | 1480344.416 |
| 0.410 | 11.480000230 | 139121.610 | 1483963.840 |
| 0.411 | 11.508000230 | 139460.931 | 1487583.264 |
| 0.412 | 11.536000230 | 139800.252 | 1491202.688 |
| 0.413 | 11.564000230 | 140139.573 | 1494822.112 |
| 0.414 | 11.692000230 | 140478.894 | 1498441.536 |
| 0.415 | 11.620000230 | 170818.215 | 1502060.960 |
| 0.416 | 11.648000230 | 141157.536 | 1505680.384 |
| 0.417 | 11.676000230 | 141496.857 | 1509299.808 |
| 0.418 | 11.704000230 | 141836.178 | 1512919.232 |
| 0.419 | 11.732000230 | 142175.499 | 1518538.656 |
| 0.420 | 11.760000240 | 142514.820 | 1520158.080 |
| 0.421 | 11.788000240 | 142854.141 | 1523777.504 |
| 0.422 | 11.816000240 | 143193.462 | 1527396.928 |
| 0.423 | 11.844000240 | 143532.783 | 1531016.352 |
| 0.424 | 11.872000240 | 143872.104 | 1534635.776 |
| 0.425 | 11.900000240 | 144211.425 | 1538255.200 |
| 0.426 | 11.928000240 | 144550.746 | 1541874.624 |
| 0.427 | 11.956000240 | 144890.067 | 1545494.048 |
| 0.428 | 11.984000240 | 145229.388 | 1549113.482 |
| 0.429 | 12.012000240 | 145568.709 | 1552732.896 |
| 0.430 | 12.040000240 | 145906.030 | 1556352.320 |
| 0.431 | 12.068000240 | 146247.351 | 1559971.744 |
| 0.432 | 12.096000240 | 146586.672 | 1563691.168 |
| 0.433 | 12.124000240 | 146925.993 | 1567210.592 |
| 0.434 | 12.152000240 | 147265.314 | 1570830.018 |
| 0.435 | 12.180000240 | 147604.635 | 1574449.440 |
| 0.436 | 12.208000240 | 147943.956 | 1578068.864 |
| 0.437 | 12.236000240 | 148283.277 | 1581688.288 |
| 0.438 | 12.264000250 | 148622.598 | 1585307.712 |
| 0.439 | 12.282000250 | 148961.919 | 1588927.136 |
| 0.440 | 12.320000250 | 149301.240 | 1592546.560 |
| 0.441 | 12.348000250 | 149640.561 | 1596165.984 |
| 0.442 | 12.386000250 | 149979.882 | 1599785.408 |
| 0.443 | 12.404000250 | 150319.203 | 1603404.832 |
| 0.444 | 12.432000250 | 150658.524 | 1607024.256 |
| 0.445 | 12.460000250 | 150997.845 | 1610643.680 |
| 0.446 | 12.488000250 | 151337.166 | 1614263.104 |
| 0.447 | 12.516000250 | 151676.487 | 1617882.528 |
| 0.448 | 12.544000250 | 152015.808 | 1621501.952 |
| 0.449 | 12.572000250 | 152355.129 | 1625121.376 |
| 0.450 | 12.600000250 | 152694.450 | 1628740.800 |
| 0.451 | 12.628000250 | 153033.771 | 1632360.224 |
| 0.452 | 12.656000250 | 153373.092 | 1635979.648 |
| 0.453 | 12.684000250 | 153712.413 | 1639599.072 |
| 0.454 | 12.712000250 | 154051.734 | 1643218.496 |
| 0.455 | 12.740000250 | 154391.055 | 1646837.920 |
| 0.456 | 12.768000260 | 154730.376 | 1650457.344 |
| 0.457 | 12.796000260 | 155069.697 | 1654076.768 |
| 0.458 | 12.824000260 | 155409.018 | 1657696.792 |
| 0.459 | 12.852000260 | 155748.339 | 1661315.616 |
| 0.460 | 12.880000260 | 156087.660 | 1664935.040 |
| 0.461 | 12.908000260 | 156426.981 | 1668554.464 |
| 0.462 | 12.936000260 | 156766.302 | 1672173.888 |
| 0.463 | 12.964000260 | 157105.523 | 1675793.312 |
| 0.464 | 12.992000260 | 157444.944 | 1679412.736 |
| 0.465 | 13.020000260 | 157784.265 | 1383032.160 |
| 0.466 | 13.048000260 | 158123.586 | 1686651.584 |
| 0.467 | 13.076000260 | 128462.907 | 1690271.008 |
| 0.468 | 13.104000260 | 158802.228 | 1693890.432 |
| 0.469 | 13.132000260 | 159141.549 | 1697509.856 |
| 0.470 | 13.160000260 | 159480.870 | 1701129.280 |
| 0.471 | 13.188000260 | 159820.191 | 1704748.704 |
| 0.472 | 13.216000260 | 160159.512 | 1708368.128 |
| 0.473 | 13.244000260 | 160498.833 | 1711987.552 |
| 0.474 | 13.272000270 | 160838.154 | 1715606.976 |
| 0.475 | 13.300000270 | 161177.475 | 1719226.400 |
| 0.476 | 13.328000270 | 161516.795 | 1722845.824 |
| 0.477 | 13.356000270 | 161856.117 | 1726465.248 |
| 0.478 | 13.384000270 | 162195.438 | 1730084.672 |
| 0.479 | 13.412000270 | 162534.759 | 1733704.096 |
| 0.480 | 13.440000270 | 162874.080 | 1737323.520 |
| 0.481 | 13.468000270 | 163213.401 | 1740942.944 |
| 0.482 | 13.496000270 | 163552.722 | 1744562.368 |
| 0.483 | 13.524000270 | 163892.043 | 1748181.792 |
| 0.484 | 13.552000270 | 164231.364 | 1751801.216 |
| 0.485 | 13.580000270 | 164570.685 | 1755420.640 |
| 0.486 | 13.608000270 | 164910.006 | 1759040.064 |
| 0.487 | 13.636000270 | 165249.327 | 1762659.488 |
| 0.488 | 13.664000270 | 165588.648 | 1766276.810 |
| 0.489 | 13.692000270 | 165927.969 | 1769898.336 |
| 0.490 | 13.720000270 | 166287.29 | 1773517.760 |
| 0.491 | 13.748000270 | 166606.611 | 1777137.184 |
| 0.492 | 13.778000280 | 166945.932 | 1780756.608 |
| 0.493 | 13.804000280 | 167285.253 | 1784376.032 |
| 0.494 | 13.832000280 | 167624.574 | 1787995.456 |
| 0.495 | 13.860000280 | 167963.895 | 1791614.880 |
| 0.496 | 13.888000280 | 168303.216 | 1795234.304 |
| 0.497 | 13.916000280 | 168642.537 | 1798853.728 |
| 0.498 | 13.944000280 | 168981.858 | 1802473.152 |
| 0.499 | 13.972000280 | 169321.179 | 1806092.567 |
| 0.500 | 14.000000280 | 169660.500 | 1809712.000 |
| 0.501 | 14.028000280 | 169999.821 | 1813331.424 |
| 0.502 | 14.056000280 | 170339.142 | 1816950.848 |
| 0.503 | 14.084000280 | 170678.463 | 1820570.272 |
| 0.504 | 14.112000280 | 171017.784 | 1824189.696 |
| 0.505 | 14.140000280 | 171367.105 | 1827809.120 |
| 0.506 | 14.168000280 | 171696.426 | 1831428.544 |
| 0.507 | 14.196000280 | 172035.747 | 1835047.968 |
| 0.508 | 14.224000280 | 172375.068 | 1838667.392 |
| 0.509 | 14.252000290 | 172714.389 | 1842286.816 |
| 0.510 | 14.280000290 | 173053.710 | 1845906.240 |
| 0.511 | 14.308000290 | 173393.031 | 1849525.664 |
| 0.512 | 14.336000290 | 173732.352 | 1853145.088 |
| 0.513 | 14.364000290 | 174071.673 | 1856764.512 |
| 0.514 | 14.392000290 | 174410.994 | 1860383.936 |
| 0.515 | 14.420000290 | 174750.315 | 1864003.360 |
| 0.516 | 14.448000290 | 175089.636 | 1867622.784 |
| 0.517 | 14.476000290 | 175428.957 | 1871242.208 |
| 0.518 | 14.504000290 | 175768.278 | 1874861.632 |
| 0.519 | 14.532000290 | 176107.599 | 1878481.058 |
| 0.520 | 14.560000290 | 176446.920 | 1882100.480 |
| 0.521 | 14.588000290 | 176786.241 | 1885719.904 |
| 0.522 | 14.616000290 | 177125.562 | 1889339.328 |
| 0.523 | 14.644000290 | 177464.883 | 1892958.752 |
| 0.524 | 14.672000290 | 177804.204 | 1896578.176 |
| 0.525 | 14.700000290 | 178143.525 | 1900197.600 |
| 0.526 | 14.728000290 | 178482.846 | 1903817.024 |
| 0.527 | 14.756000300 | 178822.167 | 1907436.448 |
| 0.528 | 14.784000300 | 179161.488 | 1911055.872 |
| 0.529 | 14.812000300 | 179500.809 | 1914675.296 |
| 0.530 | 14.840000300 | 179840.130 | 1918294.720 |
| 0.531 | 14.868000300 | 180179.451 | 1921914.144 |
| 0.532 | 14.896000300 | 180518.772 | 1925533.568 |
| 0.533 | 14.924000300 | 180858.093 | 1929152.992 |
| 0.534 | 14.952000300 | 181197.414 | 1932772.416 |
| 0.535 | 14.980000300 | 181536.735 | 1936391.840 |
| 0.536 | 15.005000300 | 181876.056 | 1940011.264 |
| 0.537 | 15.036000300 | 182215.377 | 1943630.688 |
| 0.538 | 15.064000300 | 182554.698 | 1947250.112 |
| 0.539 | 15.092000300 | 182894.019 | 1950869.536 |
| 0.540 | 15.120000300 | 183233.340 | 1954488.960 |
| 0.541 | 15.148000300 | 183572.661 | 1958108.384 |
| 0.542 | 15.176000300 | 183911.982 | 1961727.808 |
| 0.543 | 15.204000300 | 184251.303 | 1965347.232 |
| 0.544 | 15.232000300 | 184590.624 | 1968966.656 |
| 0.545 | 15.260000310 | 184929.945 | 1972586.080 |
| 0.546 | 15.288000310 | 185269.266 | 1976205.504 |
| 0.547 | 15.316000310 | 185608.587 | 1979824.928 |
| 0.548 | 15.344000310 | 185947.908 | 1983444.352 |
| 0.549 | 15.372000310 | 186287.229 | 1987063.776 |
| 0.550 | 15.400000310 | 186626.550 | 1990683.200 |
| 0.551 | 15.428000310 | 186965.871 | 1994302.624 |
| 0.552 | 15.456000310 | 187305.192 | 1997922.048 |
| 0.553 | 15.484000310 | 187644.513 | 2001541.472 |
| 0.554 | 15.512000310 | 187983.834 | 2005160.896 |
| 0.555 | 15.540000310 | 188323.155 | 2008780.320 |
| 0.556 | 15.568000310 | 188662.476 | 2012399.744 |
| 0.557 | 15.596000310 | 189001.797 | 2016019.168 |
| 0.558 | 15.624000310 | 189341.118 | 2019638.592 |
| 0.559 | 15.652000310 | 189680.439 | 2023258.016 |
| 0.560 | 15.680000310 | 190019.760 | 2026877.440 |
| 0.561 | 15.708000310 | 190359.081 | 2030496.864 |
| 0.562 | 15.736000310 | 190698.402 | 2034116.288 |
| 0.563 | 15.764000320 | 191037.723 | 2037735.712 |
| 0.564 | 15.792000320 | 191377.044 | 2041355.136 |
| 0.565 | 15.820000320 | 191716.385 | 2044974.560 |
| 0.566 | 15.848000320 | 192055.686 | 2048593.984 |
| 0.567 | 15.876000320 | 192395.007 | 2052213.408 |
| 0.568 | 15.904000320 | 192734.328 | 2055832.832 |
| 0.569 | 15.932000320 | 193073.649 | 2059452.256 |
| 0.570 | 15.960000320 | 193412.970 | 2063071.680 |
| 0.571 | 15.988000320 | 193752.291 | 2066691.104 |
| 0.572 | 16.016000320 | 194091.612 | 2070310.528 |
| 0.573 | 16.044000320 | 194430.933 | 2073929.952 |
| 0.574 | 16.072000320 | 194770.254 | 2077549.376 |
| 0.575 | 16.100000320 | 195109.575 | 2081168.800 |
| 0.576 | 16.128000320 | 195448.896 | 2084788.224 |
| 0.577 | 16.156000320 | 195788.217 | 2088407.648 |
| 0.578 | 16.184000320 | 196127.538 | 2092027.072 |
| 0.579 | 16.212000320 | 196466.859 | 2095646.496 |
| 0.580 | 16.240000320 | 196806.180 | 2099265.920 |
| 0.581 | 16.268000330 | 197145.501 | 2102885.344 |
| 0.582 | 16.296000330 | 197484.822 | 2106504.768 |
| 0.583 | 16.324000330 | 197824.143 | 2110124.192 |
| 0.584 | 16.352000330 | 198163.434 | 2113743.616 |
| 0.585 | 16.380000330 | 198502.785 | 2117363.040 |
| 0.586 | 16.408000330 | 198842.106 | 2120982.464 |
| 0.587 | 16.436000330 | 199181.427 | 2124601.888 |
| 0.588 | 16.464000330 | 199620.748 | 2128221.312 |
| 0.589 | 16.492000330 | 199860.069 | 2131840.736 |
| 0.590 | 16.520000330 | 200199.390 | 2135460.160 |
| 0.591 | 16.548000330 | 200538.711 | 2139079.581 |
| 0.592 | 16.576000330 | 200878.032 | 2142699.008 |
| 0.593 | 16.604000330 | 201217.353 | 2146318.432 |
| 0.594 | 16.632000330 | 201556.674 | 2149937.856 |
| 0.595 | 16.660000330 | 201895.995 | 2153557.280 |
| 0.596 | 16.688000330 | 202235.316 | 2157176.704 |
| 0.597 | 16.716000330 | 202574.634 | 2160796.128 |
| 0.598 | 16.744000330 | 202913.958 | 2164415.552 |
| 0.599 | 16.772000340 | 203253.279 | 2168034.976 |
| 0.600 | 16.800000340 | 203592.600 | 2171654.400 |
| 0.601 | 16.828000340 | 203931.921 | 2175273.824 |
| 0.602 | 16.856000340 | 204271.242 | 2178893.248 |
| 0.603 | 16.884000340 | 204610.563 | 2182512.672 |
| 0.604 | 16.912000340 | 204949.884 | 2188132.096 |
| 0.605 | 16.940000340 | 205289.205 | 2189751.520 |
| 0.606 | 16.968000340 | 205628.526 | 2193370.944 |
| 0.607 | 16.996000340 | 205976.847 | 2196990.368 |
| 0.608 | 17.024000340 | 206307.168 | 2200609.792 |
| 0.609 | 17.052000340 | 206646.489 | 2204229.216 |
| 0.610 | 17.080000340 | 206985.810 | 2207848.640 |
| 0.611 | 17.108000340 | 207325.131 | 2211468.064 |
| 0.612 | 17.136000340 | 207664.452 | 2215087.488 |
| 0.613 | 17.164000340 | 208003.773 | 2218706.912 |
| 0.614 | 17.192000340 | 208343.094 | 2222326.336 |
| 0.615 | 17.220000340 | 208682.415 | 2225945.760 |
| 0.616 | 17.248000340 | 209021.736 | 2229565.184 |
| 0.617 | 17.276000350 | 209361.057 | 2233184.608 |
| 0.618 | 17.304000350 | 209700.378 | 2236804.032 |
| 0.619 | 17.332000350 | 210039.699 | 2240423.456 |
| 0.620 | 17.360000350 | 210379.020 | 2244042.880 |
| 0.621 | 17.388000350 | 210718.341 | 2247662.304 |
| 0.622 | 17.41600035 | 211057.662 | 2251281.728 |
| 0.623 | 17.444000350 | 211396.983 | 2254901.152 |
| 0.624 | 17.472000350 | 211736.304 | 2258520.576 |
| 0.625 | 17.500000350 | 212075.625 | 2262140.000 |
| 0.626 | 17.528000350 | 212414.946 | 2265759.424 |
| 0.627 | 17.550003500 | 212754.267 | 2269378.848 |
| 0.628 | 17.584000350 | 213093.588 | 2272998.272 |
| 0.629 | 17.612000350 | 213432.909 | 2276617.696 |
| 0.630 | 17.640000350 | 213772.230 | 2280237.120 |
| 0.631 | 17.66800035 | 214111.551 | 2283856.544 |
| 0.632 | 17.696000350 | 214450.872 | 2287475.968 |
| 0.633 | 17.724000350 | 214790.193 | 2291095.392 |
| 0.634 | 17.752000360 | 215139.514 | 2294714.816 |
| 0.635 | 17.780000360 | 215468.835 | 2298334.240 |
| 0.636 | 17.808000360 | 215808.156 | 2301953.664 |
| 0.637 | 17.836000360 | 216147.477 | 2305573.088 |
| 0.638 | 17.864000360 | 216486.798 | 2309192.512 |
| 0.639 | 17.892000360 | 216826.119 | 231281.936 |
| 0.640 | 17.920000360 | 217165.440 | 2316431.360 |
| 0.641 | 17.940003600 | 215704.761 | 2320050.784 |
| 0.642 | 17.976000360 | 217844.082 | 2323670.208 |
| 0.643 | 18.004000360 | 218183.403 | 2327289.632 |
| 0.644 | 18.032000360 | 218522.724 | 2330909.056 |
| 0.645 | 18.060000360 | 218862.045 | 2334528.460 |
| 0.646 | 18.088000360 | 219201.366 | 2338147.904 |
| 0.647 | 18.116000360 | 219540.687 | 2341767.328 |
| 0.648 | 18.144000360 | 219880.008 | 2345386.752 |
| 0.649 | 18.172000360 | 220219.329 | 2349006.176 |
| 0.650 | 18.200000360 | 220558.650 | 2352825.600 |
| 0.651 | 18.228000360 | 220897.971 | 2356245.024 |
| 0.652 | 18.256000370 | 221237.292 | 2359867.448 |
| 0.653 | 18.284000370 | 221576.613 | 2363483.872 |
| 0.654 | 18.312000370 | 221915.934 | 2367103.296 |
| 0.655 | 18.340000370 | 222255.255 | 2370722.720 |
| 0.656 | 18.368000370 | 222594.576 | 2374342.144 |
| 0.657 | 18.396000370 | 222933.897 | 2377961.588 |
| 0.658 | 18.424000370 | 223273.218 | 2381580.992 |
| 0.659 | 18.452000370 | 223612.539 | 2385200.416 |
| 0.660 | 18.480000370 | 223951.860 | 2388819.840 |
| 0.661 | 18.508000370 | 224291.181 | 2392439.264 |
| 0.662 | 18.536000370 | 224630.502 | 2396058.888 |
| 0.663 | 18.564000370 | 224969.823 | 2399678.112 |
| 0.664 | 18.592000370 | 225309.144 | 2403297.536 |
| 0.665 | 18.620000370 | 225648.465 | 2406916.960 |
| 0.666 | 18.648000370 | 225987.786 | 2410538.384 |
| 0.667 | 18.676000370 | 226327.107 | 2414155.808 |
| 0.668 | 18.704000370 | 226666.428 | 2417775.232 |
| 0.669 | 18.732000370 | 227005.749 | 2421394.858 |
| 0.670 | 18.760000380 | 227345.070 | 2425014.080 |
| 0.671 | 18.788000380 | 227684.391 | 2428633.504 |
| 0.672 | 18.816000380 | 228023.712 | 2432252.928 |
| 0.673 | 18.844000380 | 228363.033 | 2435872.352 |
| 0.674 | 18.87200038 | 228702.354 | 2439491.776 |
| 0.675 | 18.900000380 | 229041.675 | 2443111.200 |
| 0.676 | 18.928000380 | 229380.996 | 2446730.624 |
| 0.677 | 18.958000380 | 229720.317 | 2460350.048 |
| 0.678 | 18.984000380 | 230059.638 | 2453969.472 |
| 0.679 | 19.012000380 | 230398.959 | 2457588.896 |
| 0.680 | 19.040000380 | 230738.280 | 2461208.320 |
| 0.681 | 19.068000380 | 321077.601 | 2464827.744 |
| 0.682 | 19.096000380 | 231416.922 | 2468447.168 |
| 0.683 | 19.124000380 | 231756.243 | 2472066.592 |
| 0.684 | 19.152000380 | 232095.564 | 2475686.016 |
| 0.685 | 19.180000380 | 232434.885 | 2479305.110 |
| 0.686 | 19.208000380 | 232774.206 | 2482924.864 |
| 0.687 | 19.236000380 | 233113.527 | 2486544.288 |
| 0.688 | 19.264000390 | 233452.848 | 2490163.712 |
| 0.689 | 19.292000390 | 233792.169 | 2493783.136 |
| 0.690 | 19.320000390 | 234131.490 | 2497402.560 |
| 0.691 | 19.348000390 | 234470.811 | 2501021.984 |
| 0.692 | 19.376000390 | 234810.132 | 2504641.408 |
| 0.693 | 19.404000390 | 235149.453 | 2508260.832 |
| 0.694 | 19.432000390 | 235488.774 | 2511880.256 |
| 0.695 | 19.46000039 | 235828.095 | 2515499.680 |
| 0.696 | 19.488000390 | 236167.416 | 2519119.104 |
| 0.697 | 19.516000390 | 236506.737 | 2520738.528 |
| 0.698 | 19.544000390 | 236845.058 | 2526357.952 |
| 0.699 | 19.572000390 | 237185.379 | 2529977.376 |
| 0.700 | 19.600000390 | 237524.700 | 2533596.800 |
| 0.701 | 19.628000390 | 237864.021 | 2537216.224 |
| 0.702 | 19.656000390 | 238203.342 | 2540835.648 |
| 0.703 | 19.684000390 | 238542.663 | 2544455.072 |
| 0.704 | 19.712000390 | 238881.984 | 2548074.496 |
| 0.705 | 19.740000390 | 239221.305 | 2551693.920 |
| 0.706 | 19.768000400 | 239560.626 | 2555313.344 |
| 0.707 | 19.796000400 | 239899.947 | 2558932.768 |
| 0.708 | 19.824000400 | 240239.268 | 2562552.192 |
| 0.709 | 19.852000400 | 240578.589 | 2566171.616 |
| 0.710 | 19.880000400 | 240917.910 | 2569791.040 |
| 0.711 | 19.908000400 | 241257.231 | 2573410.464 |
| 0.712 | 19.936000400 | 241596.552 | 2577029.888 |
| 0.713 | 19.964000400 | 241935.873 | 2580649.312 |
| 0.714 | 19.992000400 | 242275.194 | 2584268.736 |
| 0.715 | 20.020000400 | 242614.515 | 2587888.160 |
| 0.716 | 20.048000400 | 242953.836 | 2591507.584 |
| 0.717 | 20.086000400 | 243293.157 | 2595127.008 |
| 0.718 | 20.104000400 | 243632.478 | 2598746.432 |
| 0.719 | 20.132000400 | 243971.799 | 2602365.856 |
| 0.720 | 20.160000400 | 244311.120 | 2605985.280 |
| 0.721 | 20.188000400 | 244650.441 | 2609604.704 |
| 0.722 | 20.216000400 | 244989.762 | 2613224.128 |
| 0.723 | 20.244000200 | 245329.083 | 2616843.552 |
| 0.724 | 20.272000410 | 245668.404 | 2820482.976 |
| 0.725 | 20.300000410 | 246007.725 | 2624082.400 |
| 0.726 | 20.328000410 | 246347.046 | 2627701.842 |
| 0.727 | 20.356000410 | 246686.367 | 2631321.248 |
| 0.728 | 20.384000410 | 247025.688 | 2634940.672 |
| 0.729 | 20.412000410 | 247365.009 | 2638580.096 |
| 0.730 | 20.440000410 | 247704.330 | 2642179.520 |
| 0.731 | 20.468000410 | 248043.651 | 2645798.844 |
| 0.732 | 20.496000410 | 248382.972 | 2649418.368 |
| 0.733 | 20.524000410 | 248722.293 | 2653037.792 |
| 0.734 | 20.552000410 | 249061.614 | 2856657.216 |
| 0.735 | 20.580000410 | 249400.935 | 2660276.640 |
| 0.736 | 20.608000410 | 249740.256 | 2663896.064 |
| 0.737 | 20.636000410 | 250079.577 | 2667515.488 |
| 0.738 | 20.651000410 | 250418.898 | 2671134.912 |
| 0.739 | 20.692000410 | 250758.219 | 2674754.336 |
| 0.740 | 20.720000410 | 251097.540 | 2678373.760 |
| 0.741 | 20.748000410 | 251436.861 | 2681993.184 |
| 0.742 | 20.776000420 | 251776.182 | 2685612.608 |
| 0.743 | 20.804000420 | 252115.503 | 2689232.032 |
| 0.744 | 20.832000420 | 252151.824 | 2692851.458 |
| 0.745 | 20.860000420 | 252794.145 | 2696470.880 |
| 0.746 | 20.888000420 | 253133.466 | 2700090.304 |
| 0.747 | 20.916000420 | 253472.787 | 2703709.728 |
| 0.748 | 20.944000420 | 253812.108 | 2707329.152 |
| 0.749 | 20.972000420 | 254151.429 | 2710948.576 |
| 0.750 | 21.000000420 | 254490.750 | 2714588.000 |
| 0.751 | 21.028000420 | 254830.071 | 2718187.424 |
| 0.752 | 21.056000420 | 155169.392 | 2721806.848 |
| 0.753 | 21.084000420 | 255508.713 | 2725426.272 |
| 0.754 | 21.112000420 | 255848.034 | 2729045.696 |
| 0.755 | 21.140000420 | 256187.355 | 2732665.120 |
| 0.756 | 21.168000420 | 258526.676 | 2736284.544 |
| 0.757 | 21.196000420 | 258865.997 | 2739903.968 |
| 0.758 | 21.224000420 | 257205.318 | 2743523.392 |
| 0.759 | 21.252000430 | 257544.639 | 2747142.816 |
| 0.760 | 21.280000430 | 257883.960 | 2750762.240 |
| 0.761 | 21.308000430 | 258223.281 | 2754381.664 |
| 0.762 | 21.336000430 | 258562.602 | 2758001.088 |
| 0.763 | 21.364000430 | 258901.923 | 2761620.512 |
| 0.764 | 21.392000430 | 259241.244 | 2765239.936 |
| 0.765 | 21.420000430 | 259580.565 | 2768859.360 |
| 0.766 | 21.448000430 | 259919.886 | 2772478.784 |
| 0.767 | 21.47600043 | 260259.207 | 2776096.206 |
| 0.768 | 21.504000430 | 260598.528 | 2779717.632 |
| 0.769 | 21.532000430 | 260937.849 | 2783337.056 |
| 0.770 | 21.580000430 | 261277.170 | 2786956.480 |
| 0.771 | 21.588000430 | 261616.491 | 2790575.904 |
| 0.772 | 21.616000430 | 261955.812 | 2794195.328 |
| 0.773 | 21.644000430 | 262295.133 | 2797814.752 |
| 0.774 | 21.672000430 | 262634.454 | 2801434.176 |
| 0.775 | 21.700000430 | 262973.775 | 2805053.600 |
| 0.776 | 21.728000430 | 263313.096 | 2808673.024 |
| 0.777 | 21.756000440 | 263652.417 | 2812292.448 |
| 0.778 | 21.784000440 | 263991.738 | 2815911.872 |
| 0.779 | 21.812000440 | 264331.059 | 2819531.296 |
| 0.780 | 21.840000440 | 264670.380 | 2823150.720 |
| 0.781 | 21.868000440 | 265009.701 | 2826770.144 |
| 0.782 | 21.896000440 | 265349.002 | 2830389.568 |
| 0.783 | 21.924000440 | 265688.343 | 2834008.992 |
| 0.784 | 21.952000440 | 266027.664 | 2837628.416 |
| 0.785 | 21.980000440 | 266366.985 | 2841247.840 |
| 0.786 | 22.008000440 | 266706.306 | 2844867.264 |
| 0.787 | 22.036000440 | 267045.627 | 2848486.688 |
| 0.788 | 22.064000440 | 267384.948 | 2852106.112 |
| 0.789 | 22.092000440 | 267724.269 | 2855725.538 |
| 0.790 | 22.120000440 | 268063.59 | 2859344.960 |
| 0.791 | 22.148000440 | 268402.911 | 2862964.384 |
| 0.792 | 22.176000440 | 268742.232 | 2866583.808 |
| 0.793 | 22.204000440 | 269081.553 | 2870203.232 |
| 0.794 | 22.232000440 | 269420.874 | 2873822.656 |
| 0.795 | 22.260000450 | 289760.195 | 2877442.080 |
| 0.796 | 22.288000450 | 270099.516 | 2881061.504 |
| 0.797 | 22.316000450 | 270438.837 | 2884680.928 |
| 0.798 | 22.344000450 | 270778.158 | 2888300.352 |
| 0.799 | 22.372000450 | 271117.479 | 2891919.766 |
| 0.800 | 22.400000450 | 271456.800 | 2895539.200 |
| 0.801 | 22.428000450 | 271796.121 | 2899158.624 |
| 0.802 | 22.456000450 | 272135.442 | 2902778.048 |
| 0.803 | 22.484000450 | 272474.763 | 2906397.472 |
| 0.804 | 22.512000450 | 272814.084 | 2910016.896 |
| 0.805 | 22.540000450 | 273153.405 | 2913636.320 |
| 0.806 | 22.568000450 | 273492.726 | 2917255.744 |
| 0.807 | 22.596000450 | 273832.047 | 2920875.168 |
| 0.808 | 22.624000450 | 274171.368 | 2924494.592 |
| 0.809 | 22.652000450 | 274510.689 | 2928114.016 |
| 0.810 | 22.680000450 | 274850.010 | 2931733.440 |
| 0.811 | 22.708000450 | 275189.331 | 2935352.864 |
| 0.812 | 22.736000450 | 275528.652 | 2938972.288 |
| 0.813 | 22.784000460 | 275667.973 | 2942591.712 |
| 0.814 | 22.792000460 | 276207.294 | 2946211.136 |
| 0.815 | 22.820000460 | 276546.615 | 2949830.560 |
| 0.816 | 22.848000460 | 276885.936 | 2956449.984 |
| 0.817 | 22.876000460 | 277225.257 | 2957069.408 |
| 0.818 | 22.904000460 | 277564.578 | 2960688.832 |
| 0.819 | 22.932000460 | 277903.899 | 2964308.256 |
| 0.820 | 22.960000460 | 278243.220 | 2967927.680 |
| 0.821 | 22.988000460 | 278582.541 | 2971547.104 |
| 0.822 | 23.016000460 | 278921.862 | 2975166.528 |
| 0.823 | 23.044000460 | 279261.183 | 2978785.952 |
| 0.824 | 23.072000460 | 279600.504 | 2982405.376 |
| 0.825 | 23.100000460 | 279939.825 | 2986024.800 |
| 0.826 | 23.128000460 | 280279.146 | 2989644.224 |
| 0.827 | 23.15600046 | 280618.467 | 2993263.648 |
| 0.828 | 23.184000460 | 280957.788 | 2996883.072 |
| 0.829 | 23.212000460 | 281297.109 | 3000502.496 |
| 0.830 | 23.240000460 | 281636.430 | 3004121.920 |
| 0.831 | 23.268000470 | 284975.751 | 3007741.344 |
| 0.832 | 23.296000470 | 282315.072 | 3011360.768 |
| 0.833 | 23.324000470 | 282654.393 | 3014980.192 |
| 0.834 | 23.352000470 | 282993.714 | 3018599.616 |
| 0.835 | 23.380000470 | 283333.035 | 3022219.040 |
| 0.836 | 23.408000470 | 283672.356 | 3025838.464 |
| 0.837 | 23.436000470 | 284001.677 | 3029457.868 |
| 0.838 | 23.464000470 | 284350.998 | 3033077.312 |
| 0.839 | 23.492000470 | 284690.319 | 3036696.736 |
| 0.840 | 23.520000470 | 285029.640 | 3040316.160 |
| 0.841 | 23.548000470 | 285368.981 | 3043935.584 |
| 0.842 | 23.576000470 | 285708.282 | 3047555.008 |
| 0.843 | 23.604000470 | 286047.603 | 3051174.432 |
| 0.844 | 23.632000470 | 286386.924 | 3054793.856 |
| 0.845 | 23.660000470 | 286726.245 | 3058413.280 |
| 0.846 | 23.688000470 | 287065.566 | 3062032.704 |
| 0.847 | 23.716000470 | 287404.887 | 3065652.128 |
| 0.848 | 23.744000470 | 287744.208 | 3069271.552 |
| 0.849 | 23.772000480 | 288083.529 | 3072890.976 |
| 0.850 | 23.800000480 | 288422.850 | 3076510.400 |
| 0.851 | 23.828000480 | 288762.171 | 3080129.824 |
| 0.852 | 23.856000480 | 289101.492 | 3083749.248 |
| 0.853 | 23.884000480 | 289440.813 | 3087368.672 |
| 0.854 | 23.912000480 | 189780.134 | 3090986.096 |
| 0.855 | 23.940000480 | 290119.455 | 3094607.520 |
| 0.856 | 23.968000480 | 290458.776 | 3098226.944 |
| 0.857 | 23.996000480 | 290798.097 | 3101846.368 |
| 0.858 | 24.024000480 | 291137.418 | 3105465.792 |
| 0.859 | 24.052000480 | 291478.739 | 3109085.216 |
| 0.860 | 24.080000480 | 291816.060 | 3112704.640 |
| 0.861 | 24.108000480 | 292155.381 | 3116324.064 |
| 0.862 | 24.136000480 | 292494.702 | 3119943.488 |
| 0.863 | 24.164000480 | 292834.023 | 3123562.912 |
| 0.864 | 24.192000480 | 293173.344 | 3127182.336 |
| 0.865 | 24.220000480 | 293512.665 | 3130801.760 |
| 0.866 | 24.248000480 | 293851.986 | 3134421.184 |
| 0.867 | 24.276000490 | 294191.307 | 3138040.608 |
| 0.868 | 24.304000490 | 294530.828 | 3141660.032 |
| 0.869 | 24.332000490 | 294869.949 | 3145279.456 |
| 0.870 | 24.360000490 | 295209.270 | 3148898.88 |
| 0.871 | 24.388000490 | 295548.591 | 3152518.304 |
| 0.872 | 24.416000490 | 295887.912 | 3156137.728 |
| 0.873 | 24.444000490 | 296227.233 | 3159757.152 |
| 0.874 | 24.472000490 | 296566.554 | 3163378.576 |
| 0.875 | 24.500000490 | 296905.875 | 3166996.000 |
| 0.876 | 24.528000490 | 297245.196 | 3170615.424 |
| 0.877 | 24.556000490 | 297584.517 | 3174234.848 |
| 0.878 | 24.584000490 | 297923.838 | 3177854.272 |
| 0.879 | 24.612000490 | 298263.159 | 3181473.696 |
| 0.880 | 24.620000490 | 298602.480 | 3185093.120 |
| 0.881 | 24.668000490 | 298941.801 | 3188712.544 |
| 0.882 | 24.696000490 | 299281.122 | 3192331.968 |
| 0.883 | 24.724000490 | 299620.443 | 3195951.392 |
| 0.884 | 24.752000500 | 299959.764 | 3199570.812 |
| 0.885 | 24.780000500 | 300299.085 | 3203190.240 |
| 0.886 | 24.808000500 | 300638.406 | 3206809.664 |
| 0.887 | 24.836000500 | 300977.727 | 3210429.088 |
| 0.888 | 24.864000500 | 301317.048 | 3214048.512 |
| 0.889 | 24.892000500 | 301656.369 | 3217667.936 |
| 0.890 | 24.920000500 | 301995.690 | 3221287.360 |
| 0.891 | 24.948000500 | 302335.011 | 3224906.784 |
| 0.892 | 24.976000500 | 302674.332 | 3228526.208 |
| 0.893 | 25.004000500 | 303013.653 | 3232145.632 |
| 0.894 | 25.032000500 | 303352.974 | 3235765.056 |
| 0.895 | 25.060000500 | 303692.295 | 3239384.480 |
| 0.896 | 25.088000500 | 304031.616 | 3243003.904 |
| 0.897 | 25.113000500 | 304370.937 | 3246823.328 |
| 0.898 | 25.144000500 | 304710.258 | 3260242.752 |
| 0.899 | 25.172000500 | 305049.579 | 3253862.176 |
| 0.900 | 25.200000500 | 305388.900 | 3257481.6 |
| 0.901 | 25.228000500 | 305728.221 | 3261101.024 |
| 0.902 | 25.256000510 | 206067.542 | 3264720.448 |
| 0.903 | 25.284000510 | 306406.863 | 3268339.872 |
| 0.904 | 25.312000510 | 306746.184 | 3271959.296 |
| 0.905 | 25.310000510 | 307085.505 | 3275578.720 |
| 0.906 | 25.368000510 | 307424.826 | 3279198.144 |
| 0.907 | 25.396000510 | 307764.147 | 3282817.568 |
| 0.908 | 25.424000510 | 308103.468 | 3286436.992 |
| 0.909 | 25.452000510 | 308442.789 | 3290056.416 |
| 0.910 | 25.480000510 | 308782.110 | 3293675.840 |
| 0.911 | 25.508000510 | 309121.431 | 3297295.264 |
| 0.912 | 25.536000510 | 309460.752 | 3300914.688 |
| 0.913 | 25.584000510 | 309800.073 | 3304534.112 |
| 0.914 | 25.592000510 | 310139.394 | 3308453.536 |
| 0.915 | 25.820000510 | 310478.715 | 3311772.960 |
| 0.916 | 25.648000510 | 310818.036 | 3315392.384 |
| 0.917 | 25.676000510 | 311157.357 | 3319011.808 |
| 0.918 | 25.704000510 | 311496.878 | 3322631.232 |
| 0.919 | 25.732000510 | 311835.999 | 3326250.656 |
| 0.920 | 25.780000520 | 312175.320 | 3329870.080 |
| 0.921 | 25.788000520 | 312514.641 | 3333489.504 |
| 0.922 | 25.816000520 | 312853.962 | 3337108.928 |
| 0.923 | 25.844000520 | 313193.283 | 3340728.352 |
| 0.924 | 25.872000520 | 313532.604 | 3344347.776 |
| 0.925 | 25.900000520 | 313871.925 | 3347967.200 |
| 0.926 | 25.928000520 | 314211.246 | 3351586.324 |
| 0.927 | 25.956000520 | 314550.567 | 3355206.048 |
| 0.928 | 25.984000520 | 314889.888 | 3358825.472 |
| 0.929 | 26.012000520 | 315229.209 | 3362444.896 |
| 0.930 | 26.040000520 | 315568.530 | 3366064.320 |
| 0.931 | 26.068000520 | 315907.851 | 3369683.744 |
| 0.932 | 26.096000520 | 316247.172 | 3373303.168 |
| 0.933 | 26.124000520 | 316586.493 | 3376922.592 |
| 0.934 | 26.152000520 | 316925.814 | 3380542.016 |
| 0.935 | 26.180000520 | 317265.135 | 3384161.440 |
| 0.936 | 26.208000520 | 317604.456 | 3387780.864 |
| 0.937 | 26.236000520 | 317943.777 | 3391400.288 |
| 0.938 | 26.264000530 | 318283.098 | 3395019.712 |
| 0.939 | 26.292000530 | 318622.419 | 3398639.136 |
| 0.940 | 26.320000530 | 318961.740 | 3402258.560 |
| 0.941 | 26.348000530 | 319301.061 | 3405877.984 |
| 0.942 | 26.376000530 | 319640.382 | 3409497.408 |
| 0.943 | 26.404000530 | 319979.703 | 3413116.832 |
| 0.944 | 26.432000530 | 320319.024 | 3416736.256 |
| 0.945 | 26.460000530 | 320658.345 | 3420355.680 |
| 0.946 | 26.488000530 | 320997.666 | 3423975.104 |
| 0.947 | 26.516000530 | 321336.987 | 3427594.528 |
| 0.948 | 26.544000530 | 321686.308 | 3431213.952 |
| 0.949 | 26.572000530 | 322015.629 | 3434833.376 |
| 0.950 | 26.600000530 | 322354.950 | 3438452.800 |
| 0.951 | 26.628000530 | 322694.271 | 3442072.224 |
| 0.952 | 26.656000530 | 323033.592 | 3445691.648 |
| 0.953 | 26.684000530 | 323372.913 | 3449344.072 |
| 0.954 | 26.712000530 | 323712.234 | 3452930.496 |
| 0.955 | 26.740000530 | 324051.555 | 3456549.920 |
| 0.956 | 26.768000540 | 324390.876 | 3460169.344 |
| 0.957 | 26.796000540 | 324730.197 | 3463788.768 |
| 0.958 | 26.824000540 | 325069.518 | 3467408.192 |
| 0.959 | 26.885200054 | 325408.839 | 3471027.616 |
| 0.960 | 26.880000540 | 325748.160 | 3474647.040 |
| 0.961 | 26.908000540 | 326087.481 | 3478268.464 |
| 0.962 | 26.936000540 | 326426.802 | 3481885.888 |
| 0.963 | 26.964000540 | 326766.123 | 3485505.312 |
| 0.964 | 29.992200054 | 327105.440 | 3489124.736 |
| 0.965 | 27.020000540 | 327444.765 | 3492744.160 |
| 0.966 | 27.048000540 | 327784.086 | 3496363.584 |
| 0.967 | 27.076000540 | 328123.407 | 3499983.008 |
| 0.968 | 27.104000540 | 328462.728 | 3503602.432 |
| 0.969 | 27.132000540 | 328802.049 | 3507221.856 |
| 0.970 | 27.160000540 | 329141.370 | 3510841.280 |
| 0.971 | 27.188000540 | 329480.691 | 3514460.704 |
| 0.972 | 27.216000540 | 329820.012 | 3518080.128 |
| 0.973 | 27.244000540 | 330159.333 | 3521699.552 |
| 0.974 | 27.272000550 | 330498.654 | 3525318.976 |
| 0.975 | 27.300000055 | 330837.975 | 3528938.400 |
| 0.976 | 27.328000550 | 331177.296 | 3532557.824 |
| 0.977 | 27.356000550 | 331516.617 | 3536177.248 |
| 0.978 | 27.384000550 | 331655.380 | 3539796.672 |
| 0.979 | 27.412000550 | 332195.259 | 3543416.096 |
| 0.980 | 27.440000550 | 332534.58 | 3547035.520 |
| 0.981 | 27.468000550 | 332873.901 | 3550654.944 |
| 0.982 | 27.496000550 | 333213.222 | 3557274.368 |
| 0.983 | 27.524000550 | 333552.543 | 3557893.732 |
| 0.984 | 27.552000550 | 333891.864 | 3561513.216 |
| 0.985 | 27.580000550 | 334231.185 | 3595132.640 |
| 0.986 | 27.608000550 | 334570.506 | 3568752.064 |
| 0.987 | 27.636000550 | 334909.827 | 3572371.488 |
| 0.988 | 27.66400055 | 335249.148 | 3575990.912 |
| 0.989 | 27.692000550 | 335588.469 | 3579610.336 |
| 0.990 | 27.720000550 | 335927.790 | 3683229.760 |
| 0.991 | 27.748000550 | 336267.111 | 3586849.184 |
| 0.992 | 27.776000560 | 336606.432 | 3590495.608 |
| 0.993 | 27.804000560 | 336945.753 | 3594088.032 |
| 0.994 | 27.832000560 | 337285.074 | 3597707.456 |
| 0.995 | 27.860000560 | 337624.395 | 3901326.880 |
| 0.996 | 27.888000560 | 337963.716 | 3604946.304 |
| 0.997 | 27.916000580 | 338303.037 | 3608568.728 |
| 0.998 | 27.944000560 | 338642.358 | 3612185.152 |
| 0.999 | 27.972000560 | 338981.679 | 3615804.586 |
| 1.000 | 28.000000560 | 339321.000 | 3619424.000 |
| 1.001 | 28.02800056 | 339660.321 | 3623043.424 |
| 1.002 | 28.056000560 | 339999.642 | 3626662.848 |
| 1.003 | 28.084000560 | 340338.963 | 3630282.272 |
| 1.004 | 28.11200056 | 340676.284 | 3633880.400 |
| 1.005 | 28.140000560 | 341017.605 | 3637521.120 |
| 1.006 | 28.168000560 | 341356.926 | 3641140.544 |
| 1.007 | 28.196000560 | 341696.247 | 3644759.968 |
| 1.008 | 28.224000560 | 342035.568 | 3648379.392 |
| 1.009 | 28.252000560 | 342374.889 | 3651998.816 |

Note:
1 dalton is an atomic mass unit (a.m.u.) symbol: μ, which is conventionally assigned a value equal to one twelfth of an atom of the mass of the most abundant isotope of carbon, carbon 12. Therefore, carbon twelve is assigned an atomic mass unit, or dalton, of 12.

Embodiments of the method have been verified by laboratory testing as described in the examples herein.

In an embodiment, both heart rate and A-V nodal conduction are consistently depressed by parasympathetic nerve stimulation. Electromagnetic fields may be positioned for parasympathetic nerve stimulation by either of two methods: 1) a Helmholtz coil, five-centimeter size, surrounding the vago-sympathetic trunk dissected from the aortic sheath in the neck, or 2) via a larger, e.g., 18 inch diameter, Helmholtz coil situated on either side of the dog's chest. Or, other coil configurations may be used.

For example, in an embodiment, the system 25 (e.g., FIG. 1) is used for treatment of a subject. A subject may be placed between a first coil 31 and a second coil 33. The first coil 31 and the second coil 33 may each have a diameter of about eighteen inches (18 in) (or other sized coils may be used) and may be arranged in the familiar Helmholtz coil arrangement. Or, in some embodiments, other coil arrangements may be used. A Helmholtz coil configuration has two loops as shown in FIG. 1. A Helmholtz coil is a device that produces a highly uniform magnetic field in a space d between the first coil 35 and the second coil 37. See ROALD K. WANGSNESS, ELECTROMAGNETIC FIELDS 234 (1986).

In an embodiment, a first coil 35 may be positioned on one side of the subject's chest and the second coil 37 may be positioned on an opposite side of the subject's chest. This arrangement aligns the subject's heart along a common axis L₁-L₁. In one embodiment, the signal generator 27 used may be a Stanford Research System model D-360 ultra low distortion function generator capable of producing a frequency adjustable and an amplitude adjustable sinusoidal, rectilinear, triangular, or trapezoidal waveform input signal. In an embodiment, sinusoidal waves may be used, although rectilinear waves also provide advantages. Or, other types of waves may be used to generate the input signal.

Field strengths applied may range from nanogauss range to microgauss range. Table 3 herein provides an example of EMF fields and frequencies determined for a particular target using Jacobson Resonance (mc²=Blvq). Such fields and frequencies may vary based on the critical molecules targeted. In certain embodiments, the critical molecules may be at least one of acetylcholine; epinephrine; nor-epinephrine; serotonin; cytokines; interferon; vaso-interstinal peptide; protons; electrons; muons; mesons; and photons—sub-atomic species.

In one example embodiment, the attenuated signal from the voltage attenuator 31 may be applied to the first coil 35 and the second coil 37 for about thirty to thirty five minutes and spontaneous heart rate measured. The A-H interval may be measured during atrial pacing at a constant heart rate for three periods: prior to application of the electromagnetic radiation, during the application of the electromagnetic radiation, and for a period, e.g., 1-5 hours, 2-4 hours, or about 3 hours after the 35 minute application of the electromagnetic field. Measurements may also be made with stepwise increase in the two forms of the parasympathetic nerve stimulation mentioned above.

While the signal generator 27, the voltage attenuator 31, and the at least one inductor are shown as connected by wires, those skilled in the art recognize any means of transmitting signals between electrical components can be used. Copper or aluminum lines, circuit boards, infrared signals, or any other portion of the electromagnetic spectrum may be used to transmit signals between components.

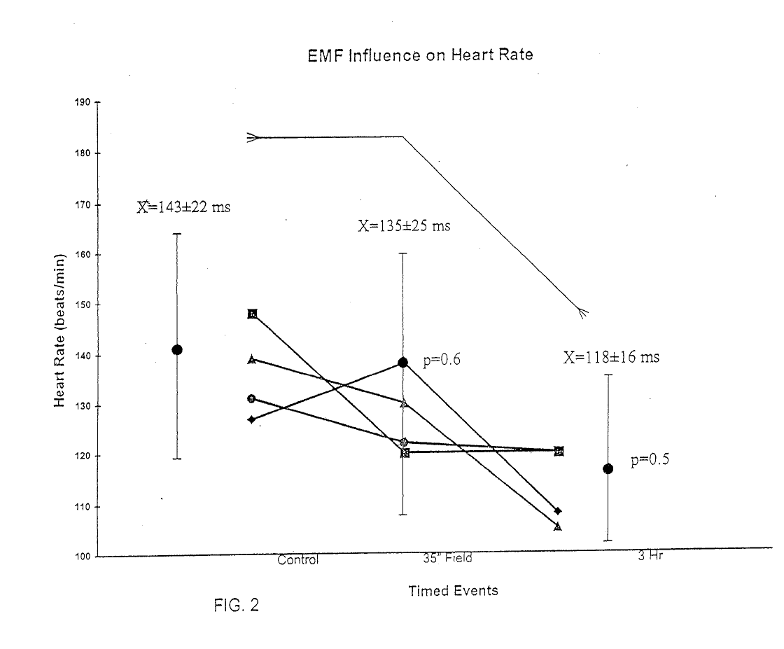

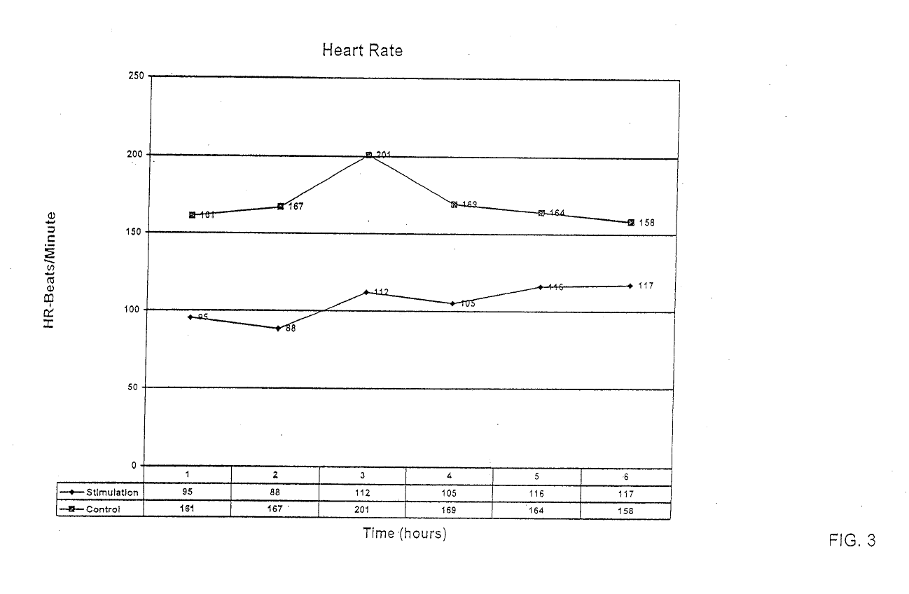

FIG. 2 graphically shows the results of the very low frequency treatment as applied to subjects (e.g., dogs) in accordance with an embodiment of the present invention. FIG. 2 shows heart rate during a three hour period after application of the electromagnetic radiation. FIG. 3 heart rate in a negative control test with no application of electromagnetic radiation. As FIG. 2 shows, in an embodiment, upon application of low-level electromagnetic fields (LL-EMF) there is a significant trend for a reduction of the spontaneous heart rate and for a reduction of the heart rate.

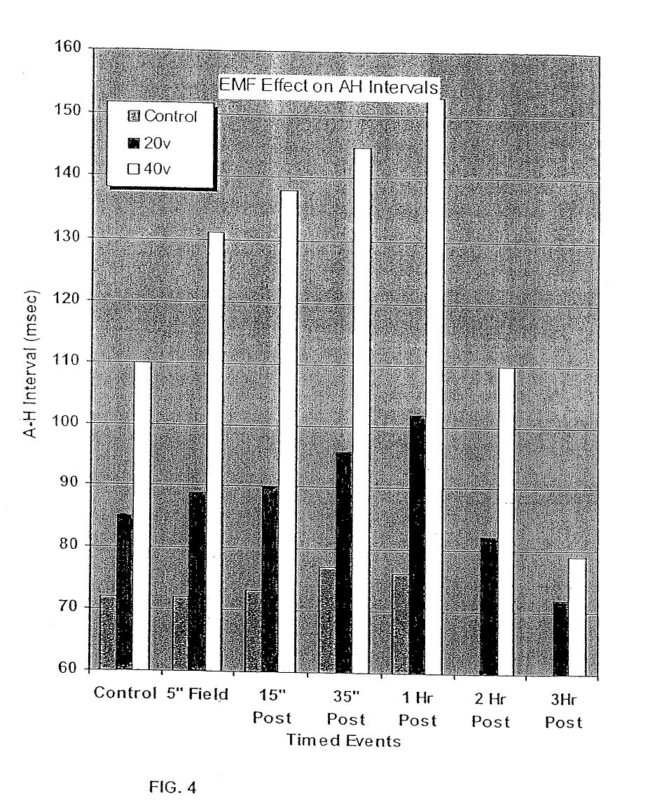

As illustrated in FIG. 4 and described in more detail in the Examples below, in an embodiment, low level EMF treatment may reduce A-H intervals in subjects.

In an embodiment, both the parasympathetic arm (slowing heart rate and A-V conduction) and the sympathetic arm (speeding heart rate and A-V conduction) arm of the autonomic nervous system could be activated by low frequency electromagnetic radiation. A balance between the parasympathetic and the sympathetic systems may result in no change in heart rate and A-V conduction, whereas, a greater sympathetic effect can induce a speeding of heart rate and A-V conduction.

In an embodiment, the parasympathetic effect may predominate despite the use of anesthesia (e.g., Na-pentobarbital). Na-pentobarbital usually affects the parasympathetic system and tends to enhance a sympathetic tone; an increased heart rate, therefore, may be experienced when Na-pentobarbital is administered. These results, however, are generally due to the greater effect of the electromagnetic field on enhancing the parasympathetic slowing of heart rate. This parasympathetic slowing of heart rate has also been seen in human patients exposed to the same low-frequency electromagnetic radiation.

In an embodiment, one direct application of the treatment is to slow heart rate. In this embodiment, the low-frequency electromagnetic treatment can activate parasympathetic neurotransmitters. This activation of parasympathetic neurotransmitters can induce slowing of the heart rate. If a subject, e.g., patient, has supraventricular tachycardias, such as the most common atrial fibrillation with a rapid ventricular response, the non-invasive application of low-frequency electromagnetic treatment can exert control over the heart rate. In this way, the treatment could provide acute control and longer term period control. Control over heart rate may be especially useful for treatment of intensive care patients, with concomitant atrial fibrillation and poor left ventricular function, in whom inotropic drugs, such as dopamine, can exacerbate rapid ventricular response. Drugs, such as beta-blockers and calcium channel blockers, may tend to slow ventricular response, but can also exacerbate heart failure and further cardiac decompensation. Cardioversion can require ventricular compromising anesthetics and, despite multiple conversions by shocks to the heart, many patients quickly revert to atrial fibrillation.

The treatment may also be applied for chronic uses. For example, in certain embodiments, the low-frequency electromagnetic treatment may be used to provide long-term “toning” of the parasympathetic nervous system. This toning may be very useful in patients with low heart rate variability. Increased parasympathetic tone has been shown to be cardio-protective in myocardial infarction survivors by increasing heart rate variability. This therapeutic modality of the present invention may be used as an adjunctive measure in patients with implantable cardioverter defibrillation (“ICD”) to reduce shock episodes, for example, by adding a coil configuration to an implanted electrode catheter. For example, a coil for “toning” the parasympathetic nervous system could be built as part of the catheter which lies in the superior vena cava adjacent to the parasympathetic nerve. This therapeutic modality could be applicable to ICD patients with and without beta-blockers, and could considerably enhance patients acceptance of ICD implantation, and significantly add to the quality of life subsequently.

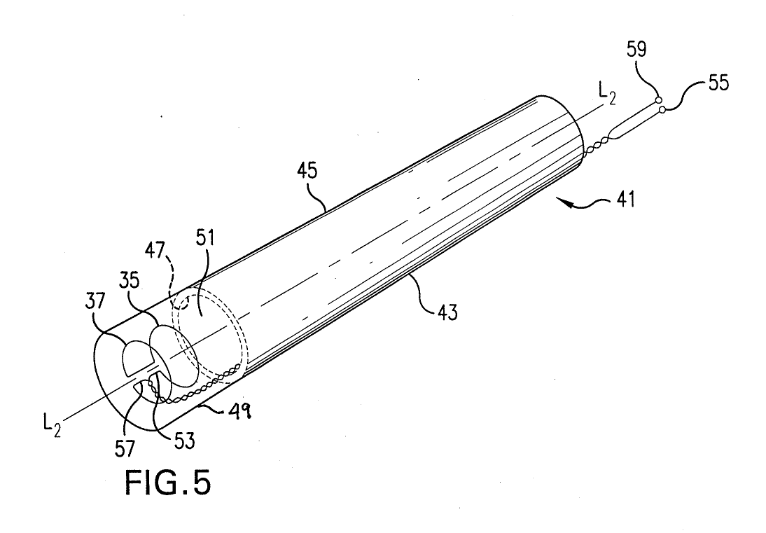

FIG. 5 shows an isometric view of a device 41 for invasively administering the low-frequency electromagnetic treatment. The device 41 includes a catheter 43. The catheter 43 is a tubular passage member 45 defining a longitudinal axis L₂-L₂. The longitudinal axis L₂-L₂ is bounded by an enclosing wall 47 to define a cross-section of the tubular passage member 45 that is transverse to the longitudinal axis L₂-L₂. The tubular passage member 45 may include a cap portion 49. The cap portion is at a distal end of the tubular passage member 45, and the cap portion securely engages the tubular passage member. At least one inductor may be contained within a bore 51 of the catheter tube 43. The inductor is shown in FIG. 5 as the first coil 35. The first coil 35 is serially arranged with the second coil 37 to produce the Helmholtz coil arrangement. As discussed previously, other coil arrangements may be substituted for the Helmholtz coil arrangement, such as solenoid or saddle coils. A first wire 53 is shown connecting the first coil 35 to a first terminal 55. This first terminal 55 receives the attenuated signal from the voltage attenuator (shown as reference numeral 31 in FIG. 1), and the attenuated signal flows through the first wire 53 and to the first coil 35. The first wire 53 connects at one end to the first coil 35, passes through the bore 51, and connects at an opposite end to the first terminal 55. A second wire 57 connects at one end to the second coil 37, passes through the bore 51, and connects at an opposite end to a second terminal 59. The second terminal 59 is connected to the signal generator 27 to complete the circuit.

The device 41 can be used to administer the low-frequency electromagnetic treatment. The device 41 may be inserted into the patient and positioned proximate a region of treatment. Once the device 41 is positioned, the attenuated signal can be sent from the voltage attenuator 31 to the at least one inductor. The attenuated signal flows through the at least one inductor and produces the magnetic flux density. The locally positioned device 41 can thus locally impinge the electromagnetic field within the patient. The device 41 allows the parasympathetic and sympathetic effects of the low-frequency electromagnetic field treatment to be focused on particular regions, or even particular organs, of the patient. The device 41, for example, could be positioned in a target region of the superior vena cava region (“SVC”) at the azagous vein junction. This particular region of treatment could interventionally reduce or increase the heart rate and the conduction rate, depending on stimulation of parasympathetic or sympathetic nervous innervation to the heart, respectively.

The catheter 43 can have a variety of configurations. Although the catheter 43 is shown as having a generally longitudinal shape, the catheter 43 may have any curvature desired to suit a particular application. One, two, three, or any number of lumina could be added for particular operations or applications. The device 41 may also include any number of ports for irrigation or suction. The specific size of the device 41 may be simply determined without undue experimentation. The size of the device 41 or any lumen may be varied to the natural conformation of the region to be treated or of the insertion passage.

The catheter 43 can also be made from a variety of materials. The catheter 43 is preferably made from a plastic material. The plastic material should have enough rigidity to be inserted into a patient, but the plastic material should also be flexible to conform to the curvature of blood vessels and organs. A guide wire may even be used to advance the to catheter for selective positioning. For example, the catheter 43 could be produced by extruding rigid polyvinyl chloride with appropriate melt characteristics for bending. Other materials include more traditional high density polyethylene, low density polyethylene, and low density polypropylene compounds.

The bore 51 of the catheter 43 can be filled with a variety of fluids. The bore 51, for example, may be exposed at a proximate end to atmospheric conditions. The bore 51, alternatively, could be filled with water, saline, dissolved oxygen, or carbon dioxide. Magneto-rheological fluids would be especially advantageous to further locally adjust the electromagnetic radiation. Any fluid compatible with the patient and with the application could be used in the bore.

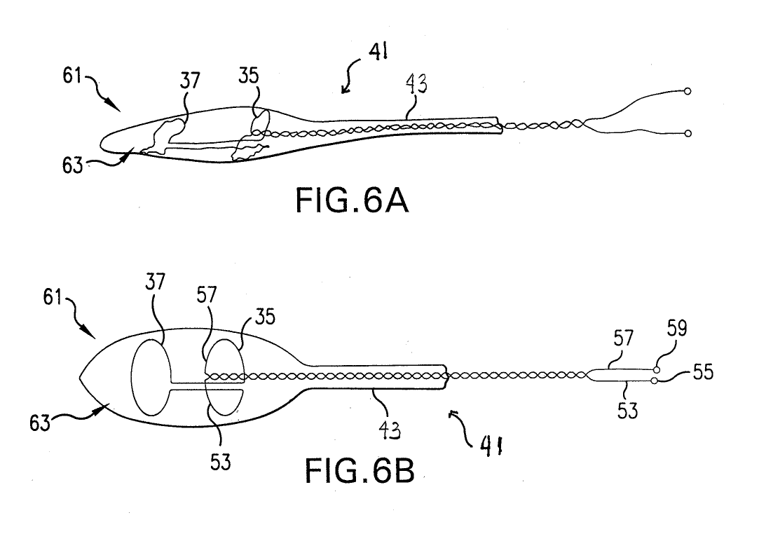

FIG. 6 includes two partial views of an alternative embodiment of a device 41 for invasively administering the low-frequency electromagnetic treatment where the device includes a balloon tip 61. FIG. 6A shows the balloon tip 61 in a deflated condition, while FIG. 6B shows the balloon tip 61 in an inflated condition. The balloon tip 61 is attached to a distal end of the catheter tube 43. The balloon tip 61 is sealed to the catheter tube 43, and an interior region 63 of the balloon tip 61 communicates with the bore of the catheter tube 43. The balloon tip 61 is inflatable and deflatable in response to fluid pressure within the bore. The balloon tip 61, for example, may be inflated by atmospheric conditions, water, saline, dissolved oxygen, carbon dioxide, or any other fluid compatible with the patient and with the application.

The balloon tip 61 may contain at least one inductor. While the inductor is shown as the first coil 35 and the serially-connected second coil 37, the inductor could include other coil arrangements discussed previously, such as solenoid or saddle coils. The inductor may be preferably small in size such that insertion of the catheter 41 into the patient is not hindered or complicated. The inductor could correspondingly expand and contract with the balloon.

The first coil 35 and the second coil 37, in this embodiment, are preferably constructed of thin wire. As illustrated in FIGS. 6A and 6B, the first coil 35 and the second coil 37 could be molded within a wall of the balloon tip 61, or the thin wire coils could be attached to the wall of the balloon tip 61. As fluid pressure within the bore of the catheter causes the balloon tip 61 to inflate, the first coil 35 and the second coil 37 would correspondingly expand and contract with the balloon.

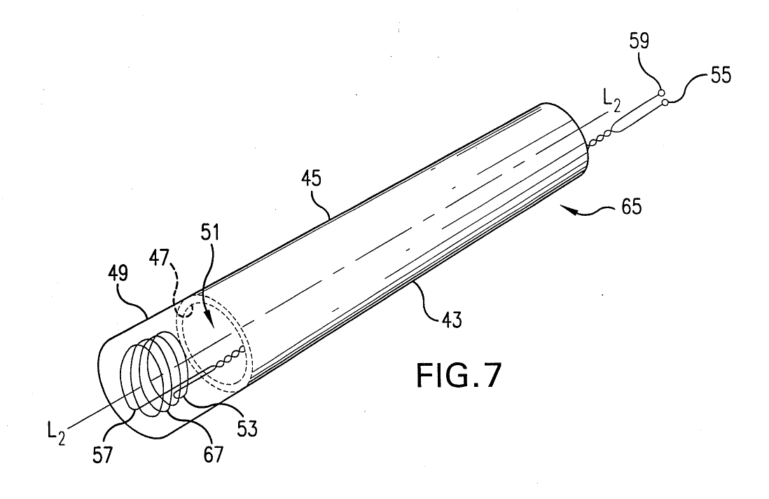

FIG. 7 is also an isometric view of an alternative embodiment of a device 65 comprising a catheter 43 for invasively administering the low-frequency electromagnetic treatment. This device 65, however, includes a solenoidal coil arrangement 67. While the solenoidal coil arrangement 67 is shown as having four (4) coils, those skilled in the art recognize the solenoidal coil arrangement 67 may consist of any number N of coils. The non-infinite length of the solenoidal coil 67, and the non-closely wound coils, ensures a constant current will produce magnetic flux density outside of the solenoidal coil arrangement 67. See DAVID K. CHENG, FIELD AND WAVE ELECTROMAGNETICS 231 (1983). The solenoidal coil arrangement 67 may be connected at one end to the first wire 53, and the solenoidal coil arrangement 67 may be connected at another end to the second wire 57. The device 65, for example, could be positioned in a target region of the SVC in the proximity of the azagous vein junction. This particular region of treatment could tone the parasympathetic nerves to the heart in patients with previous myocardial infarction (“MCI”). The treatment could prevent ventricular tachycardia and ventricular fibrillation, since enhanced parasympathetic tone has been shown to be protective against these malignant arrhythmias.

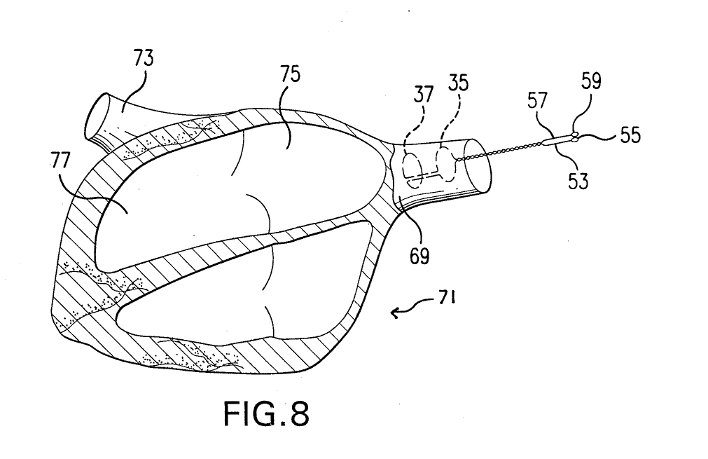

FIG. 8 shows an implantable inductor for invasively administering the low-frequency electromagnetic treatment. The implantable inductor is shown as the first coil 35 and the serially-connected second coil 37 implanted proximate the superior vena cava region 69 of a human heart 71. The inferior vena cava region 73, the right atrium region 75, and the right ventricle region 77 are shown for orientation and clarity. While the inductor is shown as the first coil 35 and the serially-connected second coil 37, the inductor could include other coil wire arrangements, such as saddle or solenoid coil arrangement (such as shown and discussed as reference numeral 67 in FIG. 7). The inductor is implantable for prevention of ventricular tachycardia by toning of the parasympathetic nerves. The Helmholtz coil arrangement of the first coil 35 and the serially-connected second coil 37, for example, could be positioned in a target region of the right ventricle. The treatment could prevent ventricular tachycardia and ventricular fibrillation in patients at risk for sudden death syndrome, due to life threatening ventricular arrhythmias.

Because the inductor is implantable, the electromagnetic treatment can be programmable. The signal generator (shown as reference numeral 27 in FIG. 1) can be configured to be implantable, and the signal generator could be programmed to periodically, randomly, or on-command, supply the input signal. A sensor could monitor parasympathetic conditions and automatically activate the signal generator. The low-frequency electromagnetic treatment can thus be applied when needed. The treatment could also be applied on-command if, for instance, the signal generator is wirelessly commanded to produce the input signal.

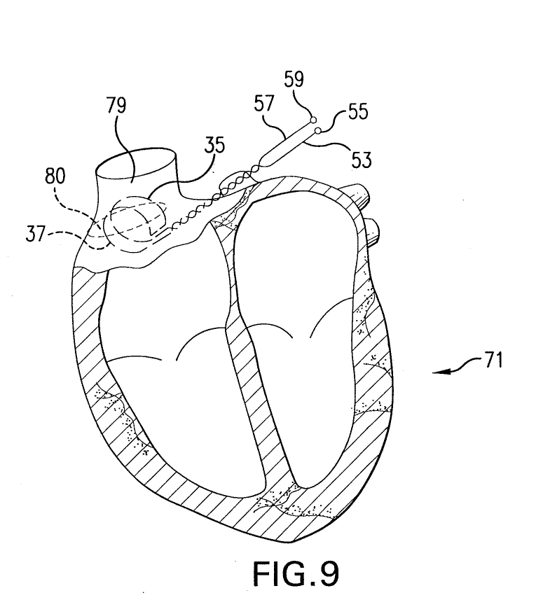

FIG. 9 shows an alternative application for the implantable inductor. The inductor is again shown as the Helmholtz coil arrangement of the first coil 35 and the second coil 37, although alternative coil arrangements may be used, such as solenoid or saddle coils. The inductor is shown implanted so as to surround the sino-atrial node region 80 near the superior vena cava 79 of the heart 71. The sino-atrial location of the inductor can focus the treatment directly on parasympathetic nerve elements at the sino-atrial node. Also, the low-frequency electromagnetic treatment may be focused on the right and left cervical vago-sympathetic nerve trunk. The low-frequency electromagnetic treatment predominantly activates the parasympathetic aim of the autonomic nervous system, and thereby can slow heart rate, A-V conduction, and reduce the rate of sinus tachycardia.

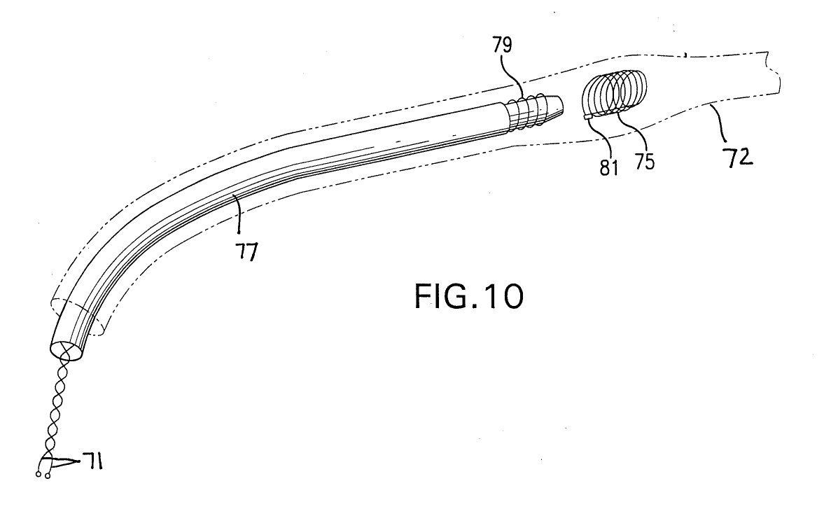

FIG. 10 also shows an alternative embodiment of a device for invasively administering low-frequency electromagnetic treatment. A stent coil 75 may be implanted in a blood vessel 72. The stent coil 75 may be a solenoid wire coil arrangement. In an embodiment, the stent coil arrangement may be implanted by standard medical devices. The coil may have a capacitor 81 attached to one end such that the solenoid 75 and the capacitor 81 are connected in series. The solenoid 75 and the capacitor 81 may thus form what is commonly referred to in the art as an “LC” circuit (“L” representing the inductor and “C” representing the capacitor.) In certain embodiments, an undriven current generated through an LC circuit will oscillate in amplitude. If there were no resistance in the LC circuit, the current could continue to oscillate indefinitely. However, there is some resistance in the LC circuit because current through a wire inherently has some resistance. The resistance of the wire has a dampening effect on the current oscillation. As with any solenoid or other wire arrangement, the oscillation of a current through the inductor coil 75 induces a magnetic field. Although the stent coil has been described as being a solenoid, other shapes to generate the field desired can be used. For example, a saddle coil may suffice.

A current may be generated in the inductor coil 75 using two methods. The first method is illustrated in FIG. 10. As shown in FIG. 10, a catheter 77 may have a solenoid coil arrangement 79 attached to one end. The catheter may be a vascular access device that is able to be inserted into a blood vessel 72. The coil 79 of wire may be attached to a generator (not shown) by wires 71. The generator sends a current through the wires 71 and the coil 79.

In one embodiment, the catheter coil 79 may be insertable into the stent coil 75. In an embodiment, if the catheter coil 79 is inserted into the stent coil 75, a current running through the catheter coil 79 will induce a current in the stent coil 75. As discussed previously, a current generated in the stent coil 75 will oscillate because the solenoid stent coil 75 and the capacitor 81 form an LC circuit. Oscillation of current amplitude is a commonly known property of LC circuits. The current in the stent coil 75 will continue to oscillate after the catheter is removed, subject to the dampening factor caused by resistivity of the wire forming the coil 75 and the capacitor 81.

The oscillation of current in the stent coil 75 can induce an electromagnetic field in the center of and around the stent coil 75. Thus, the stent coil 75 can apply an electromagnetic field locally to the subject or organism in which the stent coil 75 is implanted. The stent coil 75 can continue to apply the electromagnetic field after the catheter coil 77 is removed from insertion within the stent coil 75.

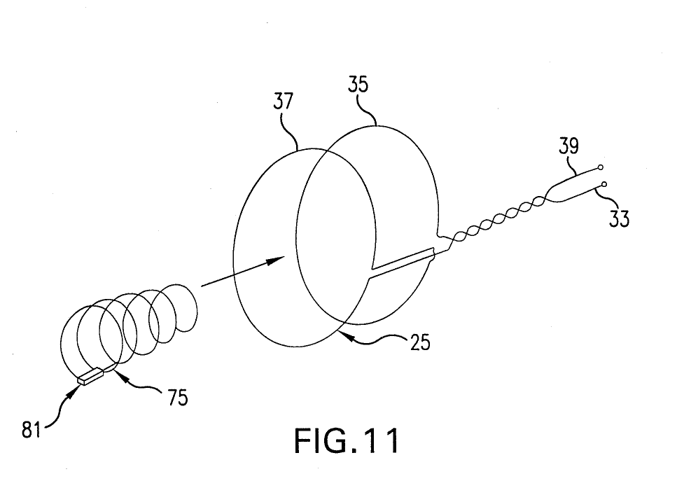

A second method of generating a current through the stent coil 75 is shown in FIG. 11. The stent coil 75 and microchip capacitor 81 may be inserted or implanted into an area of an organism such as a blood vessel in the same configuration shown in FIG. 10. The organism or subject in which the stent coil 75 has been implanted may then exposed to an electromagnetic field generated by an external coil configuration. The electromagnetic field may be generated by a Helmholtz coil configuration 25 as shown in FIG. 11. As described previously, the Helmholtz coil configuration has a first coil 35 arranged in series with a second coil 37, which is connected to a signal generator by two wires 39 and 33. Or, other coil configurations for generating an electromagnetic field may be easily substituted for the Helmholtz coil 25 arrangement shown in FIG. 11. Examples of such alternative coil arrangements include solenoid coils and saddle coils with one or more coils of a shape such that a magnetic field is induced through the coil.

The subject in which that stent coil 75 has been implanted may be placed within the magnetic field produced by the coil arrangement 25. As with the configuration shown in FIG. 10, the external magnetic field induces a current through the stent coil 75, which oscillates subject to a dampening factor. In certain embodiments, the current oscillation continues after the external magnetic field is removed.

### EXAMPLES

The following non-limiting examples use low electromagnetic field (EMF) treatment to alter cardiac functions in addition to lowering heart rate and increasing A-H intervals.

### Example 1

Preliminary studies were conducted using eight (8) anesthetized dogs. Each dog was intravenously administered 30 mg/Kg of Na-pentobarbital. The heart rates in the anesthetized state averaged 120-170 beats per minute. The baseline measurements of the heart rates were made from recordings of standard electrocardiograms. Cardiac conduction measurements were made from a His bundle electrogram. The His bundle electrogram shows conduction time from the upper chambers of the heart (the atria, A) to the beginning of electrical activation (His bundle, H) of the lower chambers (ventricles). The A-to-H interval measures conduction time in milliseconds through the A-V node.

The control measurements were recorded. Both heart rate and A-V nodal conduction are consistently depressed by parasympathetic nerve stimulation. Electromagnetic fields can be positioned for parasympathetic nerve stimulation by either of two methods: 1) a Helmholtz coil, five-centimeter size, surrounding the vago-sympathetic trunk dissected from the aortic sheath in the neck, or 2) via a larger, 18 inch diameter Helmholtz coil situated on either side of the dog's chest.

Once the control measurements were recorded, the system was used for treatment of the dogs. A dog was placed between the first coil and the second coil. The first coil and the second coil each had a diameter of eighteen inches (18 in) and were arranged in the familiar Helmholtz coil arrangement.

The first coil was positioned on one side of the dog's chest and the second coil was positioned on an opposite side of the dog's chest. This arrangement aligned the dog's heart along a common axis L₁-L₁ as shown in FIG. 1. The signal generator used in the experiments was a Stanford Research System model D-360 ultra low distortion function generator.

Field strengths applied were from nanogauss range to microgauss range in cardiovascular studies. Specific electromagnetic fields were selected on the basis of Jacobson Resonance (mc²=Blvq). The critical molecules were: acetylcholine; epinephrine; norepinephrine; serotonin; cytokines; interferon; vaso-interstinal peptide; protons; electrons; muons; mesons; and photons—sub-atomic species. Sinusoidal waves were commonly used, although rectilinear waves also provided advantages.

The attenuated signal from the voltage attenuator was applied to the first coil and the second coil for thirty five (35) minutes. Spontaneous heart rate was initially measured. The A-H interval was measured during atrial pacing at a constant heart rate for three periods: prior to application of the electromagnetic radiation, during the application of the electromagnetic radiation, and for three (3) hours after the 35 minute application of the electromagnetic field. Measurements were also made with stepwise increase in the two forms of the parasympathetic nerve stimulation mentioned above.

FIGS. 2 and 3 graphically show the results of the very low frequency treatment. FIG. 2 is a graph showing each dog's heart rate during three (3) hours after application of the electromagnetic radiation. FIG. 3 shows each dog's heart rate in a sham control test with no application of electromagnetic radiation. As FIG. 2 shows, there is a significant trend during the three (3) hours for a reduction of the spontaneous heart rate and for a reduction of the heart rate. This trend was not significant at thirty five (35) minutes, when the electromagnetic radiation was initially terminated. However, heart rates were significantly decreased at three (3) hours. FIG. 3, conversely, shows the results for the negative control during spontaneous rhythm and with electrical stimulation over a period of six (6) hours. No electromagnetic radiation was applied during the sham control, and FIG. 3 shows no trend for either an increase or a decrease in heart rate during this period.

FIG. 4 graphically shows the effects of the treatment on A-H intervals. The time for each dog's A-H interval was measured during and at several periods after the electromagnetic field application. Three autonomic nerve stimulation levels were also tested: forty volts (40V), twenty volts (20V), and a negative control (i.e., no stimulation). The rate of change of A-H prolongation or slowing of A-V conduction for one (1) to three (3) hours was greatest at the highest level (40V) of stimulation. The induction of A-V block, i.e., atrial activation not followed by ventricular activation, more interestingly occurred at the highest stimulation level (again, 40V) at two (2) and three (3) hours even though other values, of autonomic nerve stimulation were returning to control levels at three (3) hours.

These results were admittedly tempered in two dogs. One dog showed a significant increase in heart rate associated with the application of electromagnetic radiation. Another dog showed no change over the three (3) hour period. The results of these two dogs suggest perhaps both the parasympathetic arm (slowing heart rate and A-V conduction) and the sympathetic arm (speeding heart rate and A-V conduction) arm of the autonomic nervous system could be activated by low frequency electromagnetic radiation. A balance between the parasympathetic and the sympathetic systems could result in no change in heart rate and A-V conduction; whereas, a greater sympathetic effect can induce a speeding of heart rate and A-V conduction.

The parasympathetic effect is well known to predominate over the sympathetic effect. Six (6) of the eight (8) dogs, as mentioned above, experienced parasympathetic slowing of heart rate and of A-V conduction. This parasympathetic effect is pronounced despite the use of Na-pentobarbital as the anesthesia. Na-pentobarbital usually affects the parasympathetic system and tends to enhance a sympathetic tone. An increased heart rate, therefore, is usually experienced when Na-pentobarbital is administered. These results, however, are due to the greater effect of the electromagnetic field on enhancing the parasympathetic slowing of heart rate. This parasympathetic slowing of heart rate has also been seen in human patients exposed to the same low-frequency electromagnetic radiation.

### Example 2

In this example, pulsed low level EMF was applied to dissected vagal trunks or non-invasively across the chest of a subject. It was found that such pulsed low level EMF can significantly increase the threshold required to induce atrial fibrillation (AF) and can suppress AF inducibility caused by sustained AF.

In the bivagal group, two means of inducing AF were used. For one set of dogs (N=4), a means of inducing AF was used which provides high frequency stimulation (IFS) to the atria delivered during the effective refractory period of the atria so that only nerve activation could occur. It was determined that the voltage at which AF was induced was progressively increased. In the second, group a 6 hour pacing model was used to induce AF. For this group, the atria were maintained in AF by rapid pacing for 3 hours. At each hour the heart was tested when pacing was turned off, and AF ceases, by measuring the width of the AF inducibility window. It is known that there can be an incremental increase in AF inducibility during the 6 hours of pacing-induced AF (Lu et al., Circ. Arrhythmia Electrophysiol., 2008, 1:184-192). The external LL-EMF was applied after 3 hours of pacing induced AF and after the AF inducibility had substnaill increased and maintained for the next 3 hours.

In this study, for one group of subjects (n=4 dogs), dissected left and right vagal trunks were placed between two ¾ inch diameter Helmholtz coils (HCs). The HCs were attached through resistors to a function generator which induced an AC current providing an EMF of 0.034 μGauss, and a frequency 0.952 Hz, as derived from the Jacobson and Cyclotron Resonance equations, respectively, described herein. During pacing (180/min) at each site, each pacing stimulus was followed (2 ms) by a high frequency train (200 Hz, 40 msec duration) delivered during the atrial refractory period (RF). The lowest voltage that induced AF was taken as the AF threshold for that site measured at baseline and then hourly during EMF application for 3 hours (hrs). It was found that application of the low level EMF resulted in an increase in the mean AF thresholds at all sites (p<0.05).

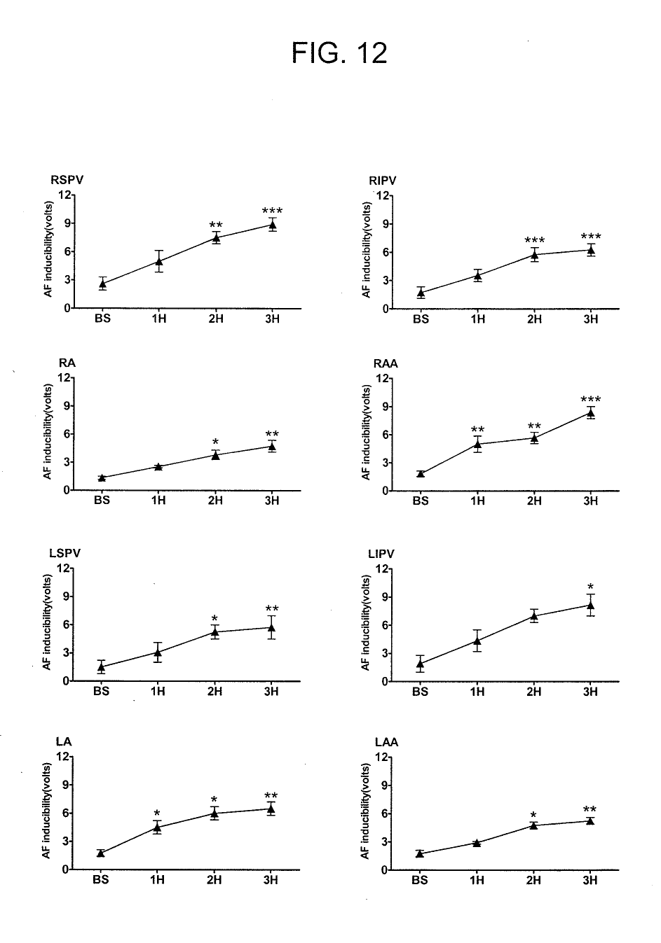

FIG. 12 illustrates the means±standard error (SE) for the four dogs (N=4) of voltage which progressively and significantly increased in response to the high frequency stimulation (HFS) that was delivered to induce atrial fibrillation (AF) at multiple sites over a 3 hr period. This response was determined at different sites in the right and left atria, including the right superior and right inferior pulmonary veins (RSPV, RIPV); the right atrial free wall and right atrial appendage (RA, RAA); the left superior and left inferior pulmonary veins (LSPV, LIPV); and the left atrial free wall and left atrial appendage (LA, LAA). AF can be induced by HFS at all these sites because of the extensive autonomic neural network that innervates the atria and ventricles (*=p<0.05; **=p<0.01; ***=p<0.001; and BS=baseline).

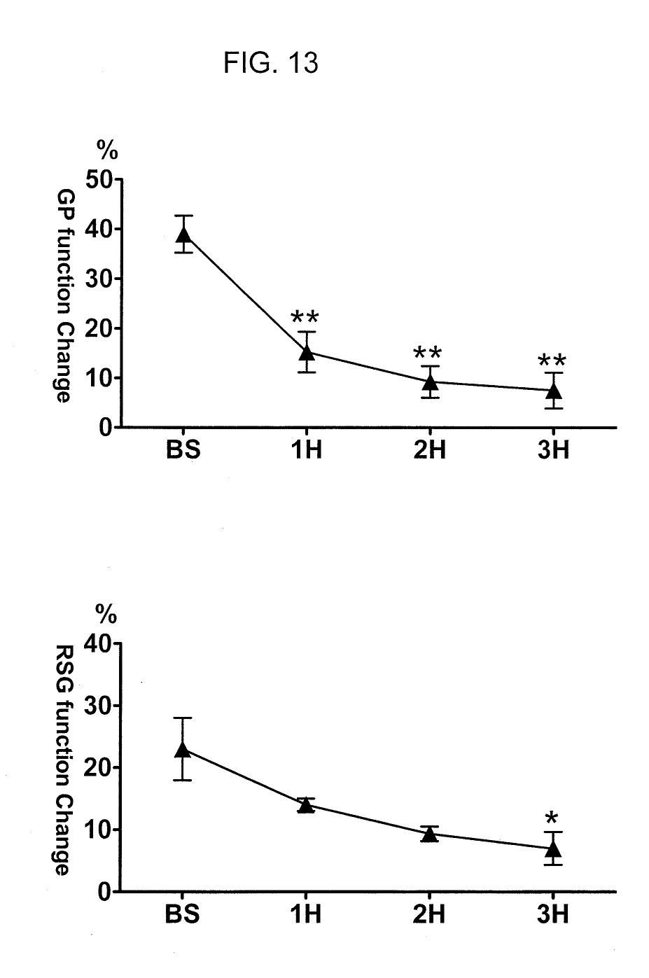

FIG. 13 (upper panel) illustrates shows the response of the ganglionated plexi (GP) to electrical stimulation over the 3 hr period during which LL-EMF was delivered to the vagal trunks. The ordinate represents the percent (%) change in the heart rate caused by applying a fixed electrical stimulation to the GP. Thus, at baseline (BS) the heart rate slowed by around 40%. During the delivery of LL-EMF there was a progressive and significant decrease in the degree to which the same stimulus delivered to the GP was able to slow the heart rate. This reduction of GP function indicates that LL-EMF may suppress autonomic activity which is the basis of AF inducibility and maintenance. For FIG. 13, *=p<0.05; **=p<0.01; ***=p<0.001; and BS=baseline.

The lower panel of FIG. 13 compares the ability of a given electrical stimulation at the right stellate ganghlion (RSG), a neural cluster which has pure sympathetic effects on the heart rate. In this case electrical stimulation of the RSG causes an increase in the heart rate around 25% faster than the normal rate at baseline and then shows a marked and progressive decrease in the percent (%) change (ability to increase the heart rate) under the influence of LL-EMF. This finding may represent a new approach to the vexing clinical syndrome known as Inappropriate Sinus Tachycardia.

In a second group (n=5 dogs), the effects of non-invasive LL-EMF were tested. An 18 inch Helmholtz Coil (HC) was positioned across the chest, so that the heart was centered within the coil. In this way, low level EMF could be administered non-invasively, using the same EMF parameters as described above for the first group of dogs. It was found that the mean effective refractory period (ERP) decreased (p<0.05) and the mean window of vulnerability (WOV) increased (p<0.001) during the first 3 hrs of induced AF compared to baseline when no low level EMF was applied. In contrast, after 3 hrs of combined EMF and induced AF, these effects were significantly reversed.

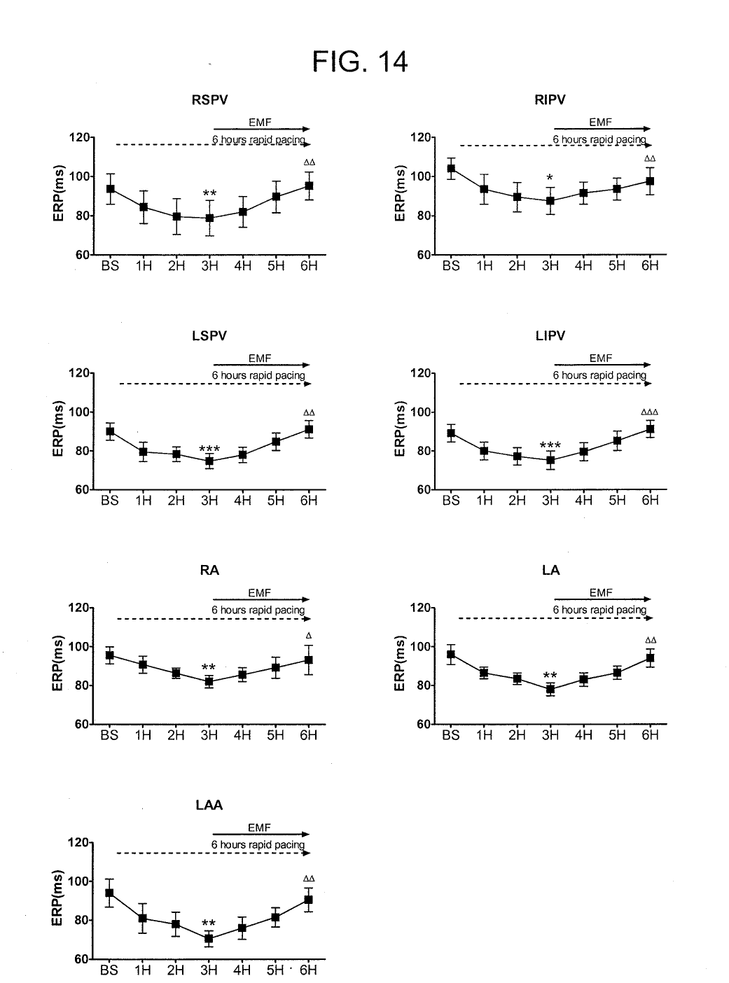

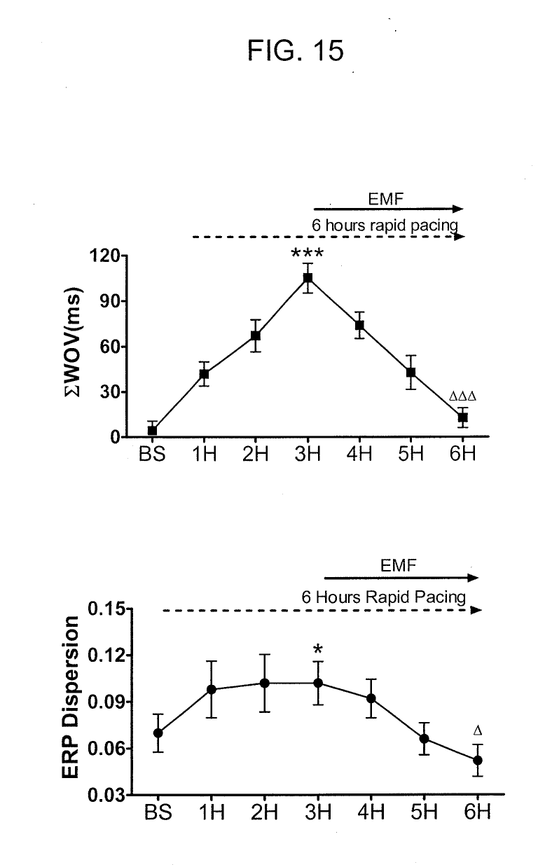

FIGS. 14 and 15 illustrate the data for this second group of dogs. FIG. 14 shows results where AF was maintained over a period of 6 hrs to show the changes in effective refractory period (ERP) of various sites (labeled as described above), both for atria and pulmonary veins, during the first 3 hrs of pacing induced AF, and then under the influence of LL-EMF applied non-invasively across the chest. It can be seen that at each site there is a progressive decrease in the ERP over the first 3 hrs and then a progressive increase toward baseline level caused by the application of LL-EMF. It is known in cardiac electrophysiology that decreasing the ERP in the atria predisposes to AF. For FIGS. 14 and 15, *=p<0.05; **=p<0.01; ***=p<0.001; Δ=p<0.05; ΔΔ=p<0.01; ΔΔΔ=p<0.001; and BS=baseline.

The upper panel of FIG. 15 shows the direct measurement of the window of vulnerability (WOV) to AF measured as the sum of the WOVs determined at all the atrial and pulmonary vein sites, the cumulative WOV (Σ WOV) as a function of the first 3 hours of pacing induced AF. It can be seen that there is a progressive and significant increase in AF inducibility and then a decrease toward baseline (BS) values after 3 hrs of LL-EMF applied non-invasively.

The lower panel of FIG. 15 shows the same pattern for ERP Dispersion which also is another measure of the propensity for AF. That is, the higher the ERP dispersion the greater the predisposition toward AF, whereas, the smaller the ERP the atria are more resistant to AF.

### Example 3

In this example, it was found that low level EMF stimulation (EMF of 0.034 microGauss, and a frequency of 0.952 Hz) of the vagus nerve (VN) can prevent and reverse autonomic remodeling, i.e., the mechanism by which sympathetic action increases induction of AF, as shown by the termination and prevention of AF inducibility.

In this study, rapid atrial pacing at 600/min was used to induce A/F, and the cumulative WOV as determined at multiple sites progressively and significantly increased after 3 hours of pacing and then returned to baseline when low level EMF was applied during the next 3 hours. However, when pacing induced AF and low level EMF vagal nerve stimulation were simultaneously applied for 6 hours, there was no significant change in cumulative WOV during that time, indicating that the low level EMF prevented autonomic remodeling.

Thus, for these experiments, ten (10) dogs (weighing 22-25 kg) were anesthetized with Na-pentobarbital. A right and left thoractomy allowed the attachment of multi-electrode catheters applied to the superior pulmonary veins (SPVs), the inferior pulmonary veins (IPVs), the atrial free walls (A), and the atrial appendages (AA). High frequency stimulation (HFS) (frequency 20 Hz) was applied bilaterally to the vagal nerves (VN) at a voltage 10% that which slowed the heart rate and/or AV conduction.

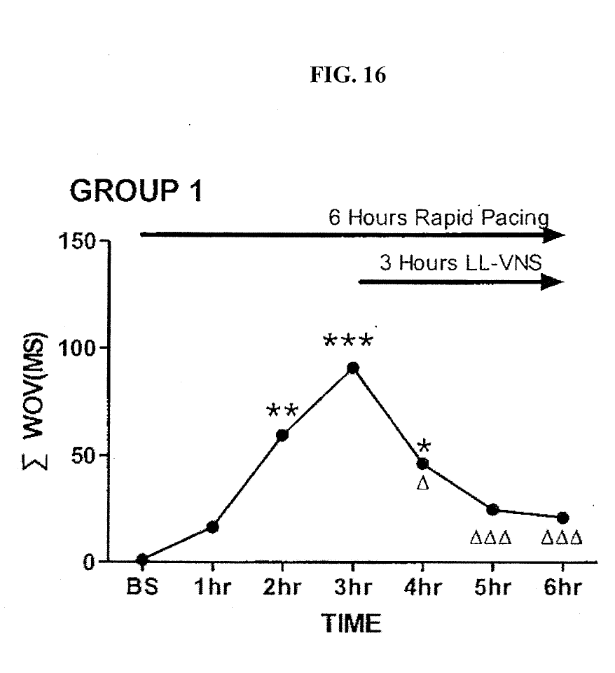

For Group 1 (n=6) (FIG. 16), programmed stimulation (PS) at 10× diastolic threshold was performed at baseline and during 6 hours of AF induced by rapid atrial pacing. Each hour, during sinus rhythm, PS consisting of S1-S1=330 ms and decremental S1-S2 allowed determination of effective refractory period (ERP) and window of vulnerability (WOV) for inducing AF. The cumulative WOV (E WOV) was the sum of the longest minus the shortest S1-S2 at which AF was induced at all test sites. Low level vagal nerve (VN) stimulation was continuously applied from the 4^{th} to the 6^{th} hour (FIG. 16).

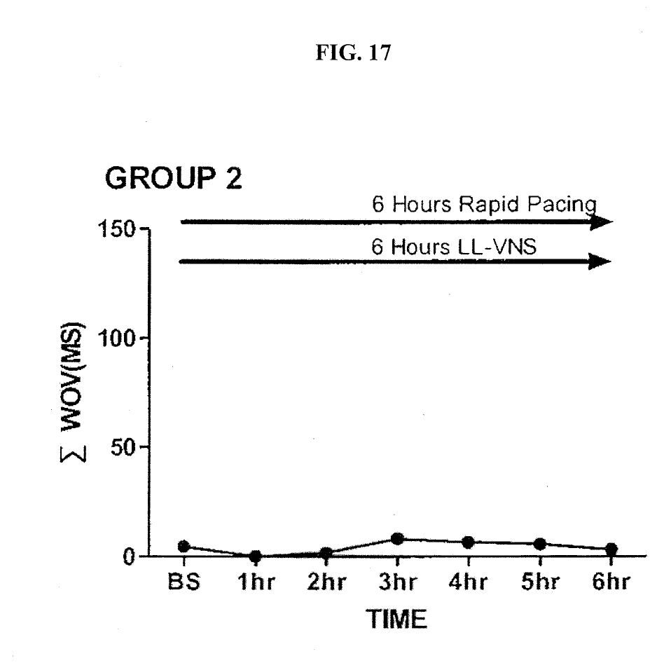

For Group 2 (n=4) (FIG. 17), after baseline determinations as described above, rapid pacing induced AF and concomitant low level VN stimulation were applied for 6 hours with hourly determinations of ERP and WOV as described above for Group 1.

It can be seen that for Group 1 (FIG. 16), the cumulative WOV (Σ WOV) progressively and significantly increased (***=p<0.0001) after 3 hours of pacing induced AF, and then returned to baseline during combined pacing induced AF and 3 hours of low level VN stimulation (AAA=p<0.0001) as compared to peak WOV. For Group 2 (FIG. 17), it can be seen that when pacing induced AF and low level VN stimulation were simultaneously applied for 6 hours, there was no significant change in cumulative WOV during that time. Thus, in sum, the data indicate that low-level EMF stimulation of the VN can prevent and reviers autonomic remodeling as shown by termination and prevention of AF inducibility.

While the present invention has been described with respect to various features, aspects, and embodiments, those skilled and unskilled in the art will recognize the invention is not so limited. Other variations, modifications, and alternative embodiments may be made without departing from the spirit and scope of the present invention. All patent and non-patent references cited herein are incorporated by reference in their entireties.

What is claimed is:

1. A method of treatment or prophylaxis of a disease state or a condition in an organism, the method comprising:
generating an electromagnetic field to be applied to the organism having a magnetic flux density (B) from about 1×10⁻⁸ gauss to about 5×10⁻⁶ gauss and a frequency of between about 0.28 Hertz to about 140 Hertz, wherein the electromagnetic field is applied therapeutically to treat or prevent cardiac diseases and conditions; and
subjecting the organism or a part thereof to the electromagnetic field to target the autonomic nervous system of the organism.

2. The method of claim 1, further comprising:
calculating the magnetic flux density (B) to be applied to the organism using the formula mc^{g}=Bvlq, wherein
m equals a mass of one or more biological targets relating to cardiac function;
c equals the speed of light;
v equals the inertial velocity of said mass;
l equals the length of the organism or cell to which the field will be applied; and
q equals unity of charge and has a value of 1 ab-coulomb.

3. The method of claim 1, further comprising administering the electromagnetic field at a location relative to the organism to target at least one of the parasympathetic or the sympathetic nervous system of the organism.

4. The method of claim 1, wherein the frequency (0 for the electromagnetic field is determined using the formula f=10 qB/(2Πm), wherein q is the charge of a particle, and m is the mass of the particle, and B is the flux density.

5. The method of claim 1, wherein the application of the magnetic field to the organism results in at least one of reducing heart rate, reducing atrial fibrillation, reducing AF-induced autonomic remodeling, and increasing A-H intervals in the organism's heart, wherein an A-H interval is the time of conduction from the atria (A) to the beginning of electrical activation of the His bundle (H) of the ventricles.

6. The method of claim 1, wherein said subjecting the organism or the part thereof to the electromagnetic field further comprises placing the organism inside an external apparatus for generating the electromagnetic field.

7. The method of claim 1, wherein said subjecting the organism or the part thereof to the electromagnetic field further comprises implanting a device for generating the electromagnetic field in the organism, wherein the apparatus is implanted in proximity to an organ to which the treatment is targeted.

8. The method of claim 1 wherein the organism is one having a diseased state or condition which is at least one of irregular heart rate, elevated blood pressure, cardiovascular failure, cancer, cataracts, immunological conditions,-blood clots, atrial fibrillation, ventricular fibrillation, atrioventricular blockage, diseased heart valves, enlarged heart, circulatory blockage, coronary insufficiencies, or ischemia.

9. The method of claim 1, wherein the electromagnetic field is administered in a range between about 10⁻⁸ gauss to about 1×10⁻⁶ gauss and a frequency between about 0.28 to about 28 Hertz to target at least one of the parasympathetic or the sympathetic nervous system.

10. The method of claim 1, wherein the electromagnetic field is in a range between about 2×10⁻⁸ gauss to about 3.8×10⁻⁸ gauss to target the parasympathetic nervous system of the organism.

11. The method of claim 10, wherein the frequency is in a range between 0.56 Hz to 1.064 Hz.

12. The method of claim 1, wherein the electromagnetic field is administered in a range between about 2.8×10⁻⁸ gauss to about 3.4×10⁻⁸ gauss to target the parasympathetic nervous system.

13. The method of claim 12, wherein the frequency is in a range between 0.854 Hz to 0.952 Hz.

14. The method of claim 1, wherein the electromagnetic field is administered in a range between about 7.5×10⁻⁸ gauss to about 1×10⁻⁶ gauss to target the sympathetic nervous system.

15. The method of claim 14, wherein the frequency is in a range between 2.10 Hz to 28 Hz.

16. The method of claim 1, wherein the target comprises at least one of vasointestinal peptide, epinephrine, serotonin, acetylcholine, tubulin subunits, adenosine, and vasostatin.
